(19) United States
(12) Patent Application Publication
(10) Pub. No.: US 2004/0018260 A1
(43) Pub. Date: Jan. 29, 2004

(54) Novel botanical extract of Tripterygium Wilfordii Hook F.

(75) Inventors: Keyong Ren, Denville, NJ (US); Xieyu Lu, Nanjing (CN); Jingjian Shen, Murrays Bay (NZ); Shangxiao Li, Parnell (NZ)

Correspondence Address: COHEN, PONTANI, LIEBERMAN & PAVANE, 551 FIFTH AVENUE, SUITE 1210, NEW YORK, NY, 10176, US

(73) Assignee: Novemed Group Limited

(21) Appl. No.: 10/174,679

(22) Filed: Jun. 19, 2002

(51) Int. Cl. A61K 35/78

(52) U.S. Cl. 424/773; 424/725

(57) ABSTRACT

A novel botanical extract from the plant Tripterygium Wilfordii Hook. F. containing bioactive components triptriolide, tripdiolide, tripchloride and tiptolide for treating inflammatory and autoimmune diseases is prepared and the bioactive components are quantified. A HPLC fingerprinting technology is also developed to produce a characteristic fingerprint for the botanical extract.

### BACKGROUND OF THE INVENTION

1. Field of the Invention

The present invention relates to a novel botanical extract of Tripterygium Wilfordii Hook. F. , which is useful for treating autoimmune and inflammatory diseases. In particular, the present invention includes detailed analysis and characterization of the novel botanical extract.

2. Description of the Related Art

Herbal medicines have emerged as a unique approach for meeting the need for safe, effective and relatively inexpensive new remedies for a variety of disorders. According to the World Health Organization (WHO), 4 billion people, i.e., about 80% of the world population, use herbal medicines for some aspects of primary health care. In the US, a recent study on trends in alternative medicine use indicated a 47.3% increase from 1990 to 1997 with 629 million total visits to alternative medicine practitioners exceeding 386 million total visits to all US primary care physicians in 1997. Herbal medicines represent the fastest growing segment among all of alternative medicine. The herbal medicines are produced in different forms, which range from crude, decocted herbs to refined, concentrated and standardized extracts. The health benefit from taking those herbals also varies with the quality of the products and the knowledge of consumers on the products. Some of the products have to be used under a physician's supervision, particularly those indicated for serious diseases although the majority of herbal medicines are generally safe. It is essential to conduct well-controlled clinical studies under guidance of regulatory authorities for herbal therapeutics to validate their safety and efficacy.

Tripterygium Wilfordii Hook. F. (TW) is a native plant in China. Roots of plant Tripterygium Wilfordii Hook. F. contains bioactive components, primarily alkaloids, diterpenes and triterpenes. A variety of preparations derived from the TW plant have been widely used in China to treat a variety of human diseases including autoimmune and/or inflammatory diseases for centuries. However, studies have shown that diterpenes are major effective components in treating rheumatoid arthritis, chronic nephritis and some other diseases. In 1972, S. M. Kupchan et al. isolated three diterpenes, namely, triptolide, tripdiolide and triptonide from the roots of Tripterygium Wilfordii Hook. F., Am. Chem. Soc. 94(20):9197, 1972. In 1979, Wu Da-Guang et al. further isolated two additional diterpenes from the same plant. Xieyu Lu et al. in 1990 reported isolation and characterization for T4 from the same plant.

Traditionally, an extract from the roots of Tripterygium Wilfordii Hook. F. (“TW extract”) containing diterpenes is used directly or to be formulated to tablets for treating patients having autoimmune diseases such as rheumatoid arthritis. Some chemical, physical and therapeutic properties of the TW extract is also described in Pharmacopoeia of the People's Republic of China” (1995 Edition). Cheng Zi Zhen et al. reported a method of obtaining a TW extract by ethanol extraction followed by ethyl acetate extraction. Chong Sao Yao, p.14, 12(5), 1981. The extract was then formulated to tablets and the total diterpene contents of each tablet were determined based on the content of triptolide (T10), which is the most abundant diterpenes in the preparation. Since triptolide (T10) has been known to induce significant toxicity, Tao Xue-Lian et al. Reported a method of obtaining a botanical extract from peeled roots of TW by sequential extraction with ethylalcohol and ethyl acetate extraction, which yields a preparation containing less triptolide. Arthritis & Rheumatism P. 130, 41(1), 1998. Since ethyl acetate extraction yields a wide range of amounts of the effective components, Xia Zhi-lin et al. recommended a method of preparing a TW extract by ethanol absent ethyl acetate. Chong Sao Yao, p. 19, 21(2), 1990. Zhang Dong Ming et al. later developed a method of determining the content tripchlorolide (T4) by HPLC. Acta Pharmaceutica Sinica, 27(8): 638-640, 1991. Chen Jun Yuan developed a method of purifiling triptolide from TW extract by a water extraction followed by an ethanol extraction, then followed by a chloroform extraction. After the chloroform extraction, the chloroform extract is subject to a sequential silica gel chromatography, eluted with chloroform and ethyl acetate-petroleum ether, respectively. The final product is obtained by re-crystalization of the ethyl acetate-petroleum eluted fractions from the silica gel column. (Pharmaceutical Industry, 1989, (5):195).

U.S. Pat. No. 5,580,562 to Lipsky et al. discloses an improved preparation of T. wilfordii (TW) root that demonstrates a more desirable therapeutic activity: toxic index ratio as compared to other preparations previously described in the literature. The therapeutic activity: toxic index ratio is calculated from an ID₅₀ in vitro T-cell proliferation/LD₅₀ ratio. According to this patent, the improved TW preparation has less than about 1.3 μg/mg triptolide, or more preferably, about 0.2 μg/mg triptolide. This improved TW preparation is preferably prepared by ethyl acetate extraction of the root or by ethanol extraction followed by an ethyl acetate extraction of the root. However, the method described by Lipsky et al. is generally considered to be crude because it is not purified by any chromatography techniques to remove undesirable components. Moreover, the concentration of triptolide in the Lipsky et al.'s preparation was not determined by HPLC.

U.S. Pat. No. 5,843,452 to Wiedmann et al. teaches a method of preparing a TW including ethanol extraction and chloroform extraction followed by silica gel column chromatography. Wiedmann et al. use methylene chloride/methanol as the elution solvent. The purified extracts thus contain no alkaloids, as determined using the Dragendorff reagent.

At least seven diterpene lactone epoxide compounds including Triptriolide (T11), Triptolide (T10), Triptolidenol (T9), Tripdiolide (T8), Triptonide (T7), Tripchlorolide (T4), and 16-hydroxy-triptolide (L2) have been identified and isolated from TW. Some in vitro and animal experiments have been performed to study the activities of each individual compound but the results are inconclusive. Among these 7 deterpene lactone epoxide compounds, T11 has the highest anti-inflammatory activity but little immunosuppressive activity, while the other six diterpene lactone epoxide compounds were found to have both anti-inflammatory and immunosuppressive activities; although T10 is the most abundant and active compound having the both anti-inflammatory and immunosuppressive activities, it is also reported to be potently carcinogenic or mutagenic; T9 and L2 are not readily obtainable due to their very low content in the plant. T7 has relatively low activities.

There has been no study whatsoever on activities of each isolated diterpene compound nor any combinations thereof in human.

While the Tripterygium Wilfordii Hook. F. extract prepared according to the traditional method(s) has been used for treating autoimmune or inflammatory diseases for many years, each diterpene content in the preparations resulting from such method(s) varies from preparation to preparation and it has never been fully analyzed and quantified. Any attempt to quantify the major bioactive components has not been satisfactory so far due to the complexity of the extract composition and technical difficulties, where multiple compounds create great interference between the components among themselves. Hence, neither physicians nor patients have had informative knowledge about the amount of active components administered to the patients, although the medicine has been used for many years. As a result of such inconsistency in the drug dosages it is difficult for physicians to monitor the treatments following prognosis of the diseases. The lack of a well defined dosage regimens also prevents this herbal medicine, that has been proven highly effective in treating autoimmune and inflammatory diseases, from being further studied for the benefits of the public at large. Thus, it is desirable to develop a standardized, quantified and reproducible form of Tripterygium Wilfordii Hook. F. extract for therapeutic uses.

Despite that various TW extracts containing diterpenes have been reported to be effective for the treatment of autoimmune and/or inflammatory diseases, but such TW extracts may be highly toxic. There has been death report resulting from administration of certain TW extract. Ttriptolide (T10) has been reported as being carcinogenic or a major component causing significant side effects, while triptriolide (T11), tripdiolide (T8) and tripchlorolide (T4) are demonstrated to be the components having the most favorable therapeutic indexes, i.e. high efficacy and low toxicity in TW extract.

The TW extracts prepared following the traditional methods usually contain a percentage of T10 that is either too high resulting in greater occurrence of side effects or too low resulting in poor efficacy. It is highly desirable to obtain a TW extract that balances between the T11, T8 and T4 contents and T10 contents so that such extract will exhibit an maximized efficacy in treating autoimmune or inflammatory diseases with minimized toxicity, i.e. an optimized therapeutic index. However, due to the complexity of the contents of an TW extract, no one has ever succeeded to obtain a TW extract that is stable, reproducible, more effective but less toxic.

### SUMMARY OF THE INVENTION

Accordingly, it is an objective of the present invention to provide an improved and standardized multiple-component form of Tripterygium Wilfordii Hook. F. (TW) extract, namely, AHT-323A botanical extract, which contains four diterpenes, i.e., triptriolide (T11), tripchlorolide (T4) and tripdiolide (T8), and triptolide (T10), in an amount defined respectively in the present application. T11, T4, T8 and T10 are major bioactive components in the AHT-323A botanical extract, which are responsible for the clinical efficacy of AHT-323A botanical extract.

According to the present invention, AHT-323A botanical extract is prepared from the dried roots of Tripterygium Wilfordii Hook. F. (“TW”) by sequential multiple steps of extractions with organic solvents followed by chromatography. The roots may be first extracted with water and/or organic solvent for several times. Preferably, the roots are extracted first with ethanol for several times then followed by extraction with chloroform for several times. The chloroform extract may then be further processed by chromatography and eluted with organic solvent such as chloroform or a combination of solvents, or by any techniques apparent to a person of ordinary skill in the art to yield a product having the substantially the same components and the same amounts of T11,, T4, T8 and T10 as exemplarily described in the present invention. Preferably, the chloroform extract is further processed by a silica gel column chromatography using a gradient of ethanol-chloroform elution solution. The eluted fractions containing T11, T4 and T8, are then collected, combined and recovered.

This AHT-323A botanic extract prepared according to the present invention was further studied and analyzed with respect to its major active components and HPLC chemical profiles—“fingerprinting”. An analytical HPLC specification for manufacturing and release tests for quality control under such specification were developed.

In one embodiment, the present invention provides a HPLC fingerprinting technology based on analysis of the HPLC profile of the AHT-323A botanical extract and a fingerprint of AHT-323A botanical extract resulting from such technology. The fingerprint comprises the isolated and identifiable peaks of T11, T4, T8 and T10 and other characteristic peaks.

In another embodiment of the present invention, AHT-323A botanical extract is characterized by an HPLC profile comprising T4, T8, T11 and T10 peaks.

In another embodiment of the present invention, AHT-323A botanical extract is characterize by an HPLC profile comprising T4, T8, T11 and T10 peaks and additional nine characteristic peaks.

In another embodiment of the present invention, Hydrocortisone is selected as an internal reference for the HPLC profile of AHT-323A botanical extract so as to provide the relative retention times for the peaks including T11, T4, T8 and T10 and the nine characteristic peaks.

In the present invention, T11, T4 and T8 are purified from AHT-323A botanical extract respectively, the amounts of which are determined by weighing the respective purified components. Thus, in another embodiment of the present invention, AHT-323A botanical extract is characterized by the amounts of T4, T8, T11 and T10, individually or in combination. More specifically, the AHT-323A botanical extract contains at least 0.09% of T11 by weight, preferably, 0.09%—about 0.18% of T11, and most preferably, about 0.18% of T11. The AHT-323A botanical extract is also characterized by at least 0.06% of T4 by weight, preferably, 0.06—about 0.13% of T4, and most preferably, 0.13% of T4. The AHT-323A botanical extract is further characterized by at least 0.018% of T8, preferably, 0.187%—about 0.036% of T8, and most preferably, 0.036% of T8. The amount of TIO in AHT-323A is preferably not more than 0.05%, and preferably, about 0.02% to about 0.04% by weight, determined by HPLC method.

In a further embodiment of the present invention, the AHT-323A botanical extract is characterized by the molar ratio of T4:T8:T11 at 0.5-1.0:0.4-0.7:0.1-0.2.

In the present invention, two of the major bioactive components of AHT-323A botanical extract, i.e. T10 and T11 are quantified by HPLC technique which is applicable for an industry scale manufacture.

The present invention provides a method to determine the amounts of T11 in AHT-323A botanical extract quantitatively by an analytical gradient HPLC, wherein the AHT-323A botanical extract sample solution is directly tested without any further purification. The amount of T11 in AHT-323A botanical extract determined by this method is at least 0.1% by weight, preferably 0.1-0.4%, and most preferably 0.4%.

Still, the present invention further provides a method to determine the amount of T10 in AHT-323A botanical extract by reversed phase HPLC, wherein the AHT-323A botanical extract sample solution is directly tested without any enrichment process. The amount of T10 in AHT-323A botanic extract determined by this method is 0.02-0.05% by weight, preferably 0.02-0.04%.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1 is a flow chart showing a preferred process of extracting AHT-323A botanical extract;

FIG. 2 is a flow chart showing a preferred process of purifying AHT-323A botanical extract;

FIG. 3 is a flow chart showing a preferred process of purifying T4, T8 and T11;

FIG. 4 is a HPLC chromatogram of purified T10;

FIG. 5 shows the linearity of T10 where the concentrations of the standard are directly proportional to the concentrations of the analyte in AHT-323A botanical extract;

FIG. 6 is a HPLC chromatogram of T10 in AHT 323A botanical extract showing the concentration of T10;

FIG. 7 is a HPLC profile of purified T11 as a reference for the fingerprint of AHT-323A botanical extract;

FIG. 8 is a HPLC profile of purified T8 as a reference for the fingerprint of AHT-323A botanical extract;

FIG. 9 is a HPLC profile of purified T10 as a reference for the fingerprint of AHT-323A botanical extract;

FIG. 10 is a HPLC profile of purified T4 as a reference for the fingerprint of AHT-323A botanical extract;

FIG. 11 is a HPLC profile of the four bioactive components T4, T8, T10 and T11;

FIG. 12 is a HPLC profile of hydrocortisone as a reference for the four bioactive components;

FIG. 13 is a HPLC fingerprint of AHT-323A botanical extract;

FIG. 14 is an expanded chromatogram to show the resolution of T11 in AHT-323A botanical extract;

FIG. 15 is another version of the HPLC fingerprint of AHT-323A botanical extract where AHT-323A botanic extract sample is 2 mg/ml at an injection volume of 50 μl;

FIG. 16, is a HPLC chromatogram of AHT-323A botanical extract spiked with hydrocortisone; and

FIG. 17 shows a result of TLC method for separating T4, T8, T11 and T10.

### DETAILED DESCRIPTION OF THE PRESENTLY PREFERRED EMBODIMENTS

As used herein, the term “AHT-323A botanical extract” refers to a Tripterygium Wilfordii Hook. F. (“TW”) extract that has, but not limited to, the features characterized in the present application.

Tripterygium Wilfordii Hook. F. extracts or TW extracts refer to any TW extracts prepared following traditional methods and used as a traditional Chinese folk medicine.

T4 is tripchlorolide having the following structure:

[symbol]

T11 is tripriolide having the following structure:

[symbol]

T8 is tripdiolide having the following structure:

[symbol]

T10 is triptolide having the following structure:

[symbol]

The term “fingerprint of AHT-323A botanical extract” means the analytical HPLC profile of AHT-323A extract.

The term “biomarker” means the active component that is representative in AHT-323 botanical extract with respect to the extract's quality.

RRT is the abbreviation of “relative retention time”.

HPLC is the abbreviation of “high performance of liquid chromatography”.

TLC is the abbreviation of “thin layer chromatography”.

RSD is the abbreviation of “relative standard deviations”

LC-MS is the abbreviation of “liquid chromatography-mass spectrometry”

I. Preparation of AHT-323 Botanical Extract

AHT-323A botanical extract is generally prepared according to the following method, which is designed to maximize the therapeutic contents of T11, T4 and T8 in the extract, but to minimize the toxic content of T10:

The dried roots of Tripterygium Wilfordii Hook. F. (“TW”), are collected in P. R. China, examined and separated from other parts of the same plant or any foreign plants or contaminants prior to extraction. The roots of TW are then cut into small pieces, approximately 2×3 cm each, by any suitable tools such as a knife or a machine.

After cutting, the small pieces of the TW roots are loaded into an extractor that is commonly used in the industry and extracted with an industrial-grade alcohol, preferably, ethanol, for 3-5 times.

The supernatants from the alcohol extractions are combined and transferred to a recovery tank, which is commonly used in the industry. The alcohol is then recovered at room temperature under reduced pressure and an alcohol liquid extract of TW roots is thus obtained.

The alcohol extract so obtained is transferred to a large-scale chemical extractor commonly used in the industry, added with an adequate amount of industrial-grade chloroform at a volumetric ration of 3-6:1 (chloroform to extract), dissolved completely, and then filtered. The filtrate is collected in a storage tank. The remaining material was re-extracted with chloroform and filtered. The filtrate was added to the storage tank. This process was repeated (not more than four times) until the filtered extract appeared colorless under visual examination. The chloroform was then recovered and a dried crude powder extract was obtained by drying under heat and reduced pressure.

The crude powder from the chloroform extraction was then loaded (at a ratio of the crude extract to silica gel equaling to 1:10) onto a large-scale production silica gel columns that have been equilibrated with chloroform. The column was then eluted stepwise with chloroform solutions containing a gradient of ethanol ranging from 0.5-30%, respectively. The elution fractions were collected into cylindrical stainless steel containers.

The elution fractions were tested for its chemical constituents of diterpenoid compounds by TLC. The fractions showing positive for T4, T8 and T11 components were combined and concentrated via recovery of chloroform solvent at room temperature under a reduced pressure. The chloroform fluid extract was then obtained. To remove chloroform residue, the fractions were dissolved in ethanol, mixed and dried under heat and reduced pressure. This process may be repeated.

The dried extract, namely, AHT-323A botanical extract so produced may be ground to fine powder for further analysis.

The above described process yields about 0.15-0.30% AHT-323A botanical extract by weight, i.e., about 1.5-3 kilograms of AHT-323A botanical extract powder may be obtained from about 1000 kilograms of dried TW roots.

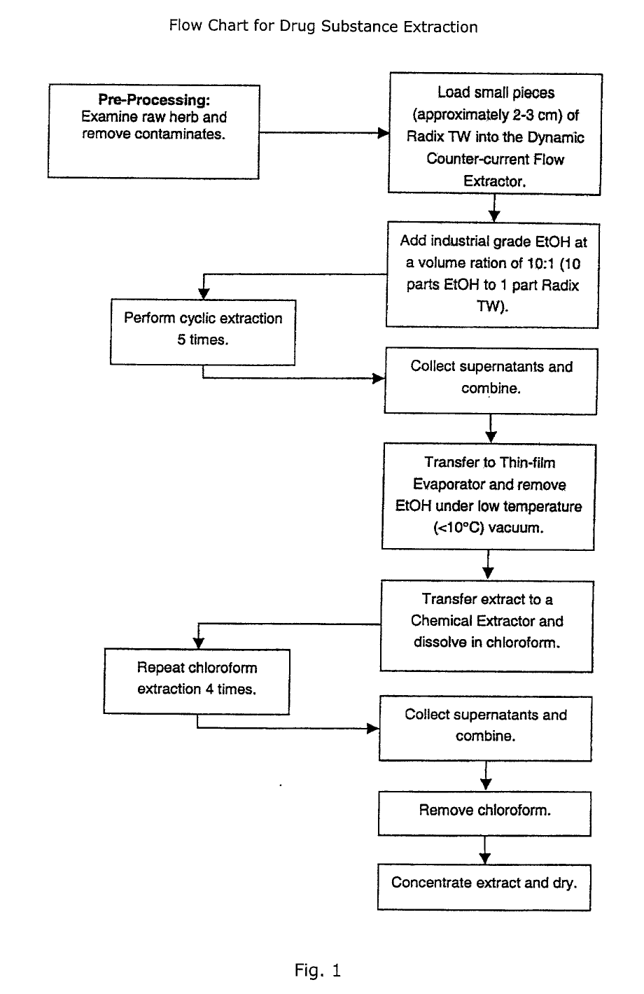

FIG. 1 is a flow chart, which schematically shows a preferred process of the preparation of AHT-323A botanic extract.

To determine the amount of T11, T4 and T8, each of these three compounds were isolated and purified. The amount of T10 in AHT-323A botanic extract was determined by a novel reversed phase HPLC methodology, wherein the AHT-323A botanical extract sample solution is directly tested without further purification process. Purified T10 was used as the standard.

II. Purification of T11, T4 and T8 and Determination of the Amounts Thereof

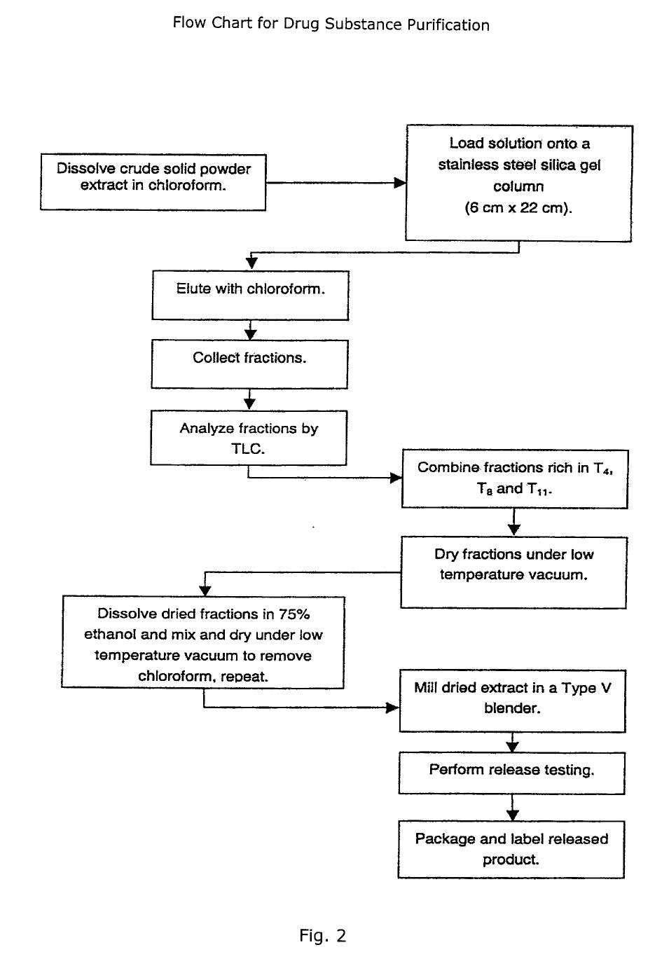

The diterpene single compounds T11, T4, and T8 are isolated and purified from the AHT-323A botanical extract as shown schematically in FIG. 2. The extract was processed mainly by flash column chromatography packed with various media, such as, silica gel, Diol and reverse phase adsorbents (C₁₈), and gel permeation chromatography. The isolations were monitored using TLC (sprayed with Kedde reagent), HPLC and LC-MS. The individual marker compounds were finally purified by recrystallization or preparative HPLC in coupled with recrystallization from various solvents. The purified compounds were identified by LC-MS and by comparing their ¹H and ¹³C data with those from literature. However, the structure of triptriolide was elucidated from a detailed analysis of its 1D and 2D NMR (including COSY, HMQC and HMBC) data due to limited data available in the literature. In addition, a TLC chromatogram and Rf values have also been evaluated.

Preferred Materials and Conditions for Quantification of T11, T4 and T8

TLC was conducted on precoated Kieselgel 60 F₂₅₄ (product code: 1.05554; Merck) or on precoated DIOL F₂₅₄_{^{S}} HPTLC (Art. 12668; Merck) and the spots were detected by spraying Kedde reagent. All LH-20 column chromatography was carried out on a glass column (2.8×180 cm) with 300 g Sephadex LH-20. The silica used for flash column chromatography is SDS SILICE 60 AC.C 40-63 μm; C18 for reverse phase flash chromatography is Sepra 55 μm C18e 70A Bulk Media (Part No: 04k-4348, Phenomenex). The mobile phase used with reverse phase flash chromatography and silica flash chromatography for separation of T11 from CK₃ is 30% to 100% MeOH/water and 12% to 20% acetone/CH₂Cl₂, respectively. A column (1.6×22 cm) packed with 20 g of DIOL (IST 60A, 40-70 μm; International Sorbent Technology Ltd) was used for all flash DIOL column chromatography. A Waters 600 HPLC machine equipped with PDA detector and a Prep Nova HR C18 column (7.8×300 mm, part no: WAT 025820) was used for final purification work and purity testing. LC-MS analysis was carried out on a HP 1100 LC/MSD system with a ZORBAX Eclipse XDB-C8 column (4.6×150 mm, 5 μm) attached. The mobile phase used for the analysis is 30% ACN/50 mM ammonium formate (PH4.5) in isocratic mode. The mobile phase flow rate is 0.5 ml/min. Drying gas flow is 10 L/min, drying gas temperature is 300° C., nebulizer pressure is 25 psig. A fragmentor voltage of 70V was applied for acquiring TIC signals. The capillary voltage was set at 3500V (positive). The injecting volume of the sample (at a concentration of 100 mg/ml) for LC-MS analysis is 20 μl. Proton and carbon NMR spectra were recorded on Bruker AC-300 spectrometer.

TLC Analysis:

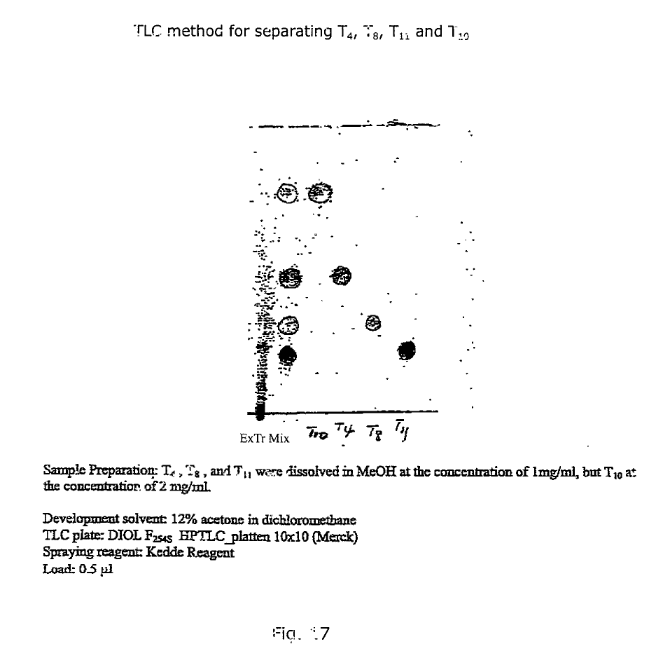

FIG. 17 is a result of TLC analysis. The Rf value and the conditions for performing this analysis are described as follows.

| Compound name | Rf value |
|---|---|
| T10 | 0.76 |
| T4 | 0.48 |
| T8 | 0.30 |
| T11 | 0.19 |

Sample preparation: T4, T8 and T11 were dissolved in MeOH at the concentration of 1 mg/ml, but T10 at the concentration of 2 mg/ml.

Developing solvent: 12% acetone in dichloromethane

TLC plate: DIOL F₂₅₄ₛ HPTLC-Platten 10×10 cm (Merck)

Spraying reagent: Kedde reagent.

Load: 0.5 μl

It is apparent to a person of ordinary skill in the art that the equipment, instruments, reagents and other materials used in the following test or determination are not limited to those specified in the following described embodiments and those equipment, instruments, reagents and other materials are generally available to a person of ordinary skill in the art.

Preferred Process of Purification of T4, T8 & T11

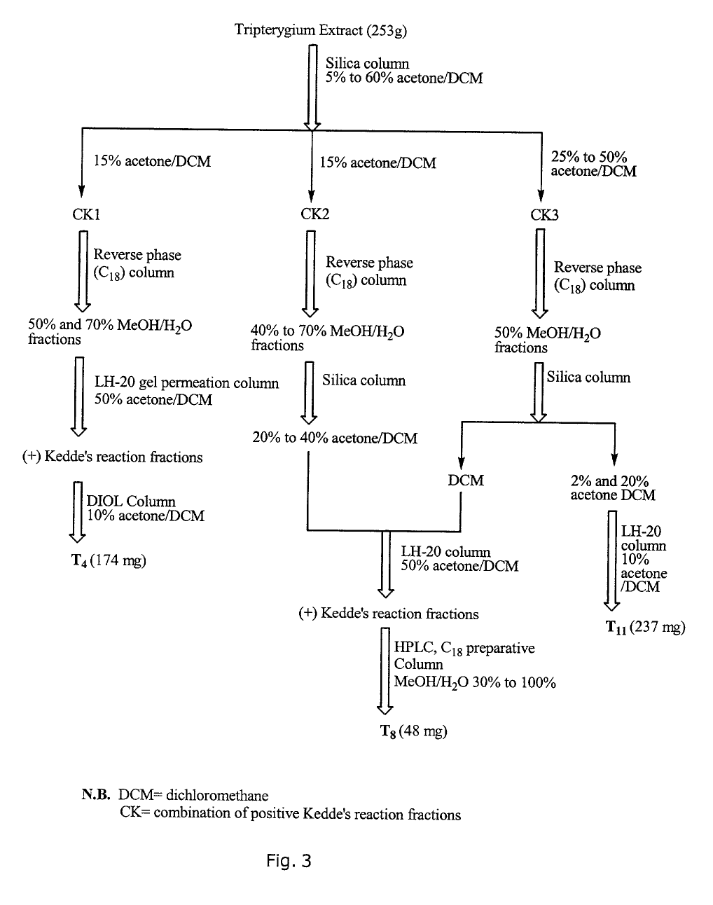

Referring now to FIG. 3, which is a flow chart of a preferred purification process for T4, T8 & T11, about 253 grams of the AHT-323A botanical extract prepared as described above was put in 30% acetone in dichloromethane the day before the silica column (100 g, 11.5×40 cm) chromatography. The solution was adsorbed on celite and evaporated to give a powder which was loaded onto the column, which was eluted with 5% to 70% acetone in dichloromethane in a stepwise mode. The fractions showing positive reaction to Kedde reagent were combined into groups CK₁, CK₂ and CK₃ according to both TLC and LC-MS analysis results.

Still referring to FIG. 3, CK₁ was subjected to reverse phase (C₁₈) column (220 g, 5×60 cm) chromatography and eluted with a gradient of MeOH/water. The fractions eluted by 50% and 70% MeOH were dried on a Speed Vac Plus vacuum drying system. The residue was passed through a column packed with Sephadex LH-20 (trade name for a product of Pharmacia Co.). 50% acetone/CH₂Cl₂ was used for elution. The positive Kedde's reaction fractions were further processed on a Diol column to give 174 mg of tripchlorolide (T4) colorless needles after recrystallization in MeOH.

Still referring to FIG. 3, CK₂ which mainly contains tripdiolide (T8), was subjected to reverse phase (C₁₈, 220 g, 5×60 cm) and silica column (76 g, 3×60 cm) chromatography, and eluted with a gradient of MeOH/water to MeOH/CH₂Cl₂ (1/1) and a gradient from 10% to 40% acetone/CH₂Cl₂ respectively, before combination with a fraction from CK₃. The combined fraction was passed though a LH-20 column with 20% acetone/CH₂Cl₂ as eluent; 48 mg of T8 was obtained by repeated preparative HPLC using methanol-water mixture with gradient pattern, then recrystallization in a mixture of MeOH/CHCl₃.

Still referring to FIG. 3, fraction CK₃ was processed with the same reverse phase (C₁₈) and silica column chromatography using similar elution solvents as those used for CK₂. Dichloromethane eluted fractions from the silica column were combined with those containing T8 from CK₂. Fractions eluting at 12% and 20% acetone/CH₂Cl₂ were loaded onto a LH-20 column, which was eluted with 10% acetone/CH₂Cl₂. Kedde's reaction positive fractions were repeatedly crystallized in a mixture of MeOH/CH₂Cl₂ to produce 237 mg of prismoid T11 (triptriolide).

The purity of these three purified compounds was assessed by RP HPLC with a normalization method. A purity of 95.6%, 98.6% and 98.3% was found for tripchlorolide (T4), tripdiolide (T8) and triptriolide (T11), respectively. LC-MS with scan mode was also used for purity testing of these compounds. The profile of TIC signals is similar to the one obtained by PDA detector. In addition, the crude extract was analyzed by LC-MS with SIM mode. The retention time found for T4, T8 and T11 was 5.0, 5.9 and 15.6 minutes, respectively.

Chemical and Physical Properties of T4, T8, and T11

Tripchlorolide (T4): colorless needles; mp 234.8-236.7° C., UV (ACN/water) λₘₐₓ 220 nm; a purple color was yielded from Kedde's reaction. LC-MS (ES) shows MH⁺, MNH₄⁺, MNa⁺ and MK⁺ at m/z 397, 414, 419 and 435 respectively leading to the molecular weight of 396. Except one of the H-1 protons in ¹H spectrum, all the ¹H and ¹³C chemical shifts are consistent with those of T4 reported in the literature. The signal at δ1.57 (CDCl₃) was assigned to one of the protons attached to C-1 based on both 1D and 2D NMR spectra (see Table1 and Table 2). To make a comparison of NMR data easier, completely assigned ¹H NMR data acquired in DMSO-d₆, which were not found in the literature, were also included in the present application. The above analysis indicates that the isolated compound is same as the tripchlorolide previously reported in the literature.

Triptriolide (T11): colorless prismoid crystals; mp 237.0-241.1° C., UV (ACN/water)λₘₐₓ 220 nm; a purple color was formed from Kedde's reaction. LC-MS (ES) shows MH⁺, MNH₄⁺, MNa⁺ and MK⁺ at m/z 379, 396, 401 and 417 respectively corresponding to the molecular weight of 378. This compound was identified as triptriolide based on careful comparison of the ¹H and ¹³C NMR data of the isolated one with those found in the literature (see Table3 and Table4). The NMR experiments carried out in this project indicated that DMSO-d₆ is a better solvent for acquiring both ¹H and ¹³C NMR spectra, thereby removing the need for solvent change in different NMR experiments.

Tripdiolide (T8): colorless crystals; UV (ACN/water) λₘₐₓ 217 nm; Kedde's reaction gave a purple colored spot. A molecular weight of 376 was deduced from the observation of ions at m/z 377, 394, 399 and 415 corresponding to MH⁺, MNH₄⁺, MNa⁺ and MK⁺ respectively in a LC-MS (ES) spectrum. The structure was elucidated by detailed analysis of both 1D and 2D NMR spectra due to sparse NMR data available to the author.

A comparison of ¹H spectra between the compound and T11 indicated that the former has two hydroxyl signals and a new signal at δ4.33, but the latter has three hydroxyl signals. Chemical shift deviations at H-11, H-12 and H-14 were also observed, which suggested that either the functional groups attached directly to these carbons or to the nearby carbons were changed. A new doublet at δ58 was also was found in ¹³C NMR spectrum of the compound. The hydroxyl signal at δ5.1 was assigned to C-2 because it correlates to H₂, which shows correlations both to H₁ and H₁₉. This assignment was confirmed by the observation of the correlations between the hydroxyl proton and C-1, C-2 and C-3 in a HMBC experiment. H-14 coupled only with its gem-hydroxyl proton at δ4.63. However, both protons showed long-range heteronuclear couplings with C-13, while H-14 also coupled with C-12. The couplings between H-11 and H-12 were readily identified in a COSY spectrum. Their relative positions were established with a HMBC experiment. Observations of a two bond correlation to C-9 and a three bond correlation to C-10 put the proton at δ3.89 at C-11. Correlation from both H-11 and H-14 to C-12 at δ54.5 further confirmed the assignment. Two hydroxyl groups in the structure of T11 replacing an epoxy subunit between C-12 and C-13 can reasonably explain the chemical shift differences at C-11, C-12 and C-14 between these two compounds. The third compound was identified as tripdiolide. Completely assigned ¹H and ¹³C NMR data are given in Table 5 and Table 6.

TABLE 1
¹H-NMR Spectral Data of T4*

| | T4 (CDCl₃, | | |
|---|---|---|---|
| Proton | Literature)³ | T4 (CDCl₃) | T4 (DMSO-d₆) |
| 1-H₂ | 1.16, 1.27 | 1.57, 1.17 | 1.41 (dd, J=12.52, |
| | | | 4.55) |
| | | | 1.26 (m) |
| 2-H₂ | 2.17, 2.32 | 2.27 (m), 2.09 | 2.14, 1.99 |
| 5-H | 2.75 (dd) | 2.65 (dd, J=6.84, | 2.67 (d, J=13.63) |
| | | 6.87) | |
| 6H_{α} | 2.20 (m) | 2.08 (m) | 2.19 (m) |
| 6H_{β} | 1.98 (m) | 1.94 (M) | 1.86 (m) |
| 7-H | 3.45 (d, J₇,_{6α}=5.86) | 3.33 (d, J=5.65) | 3.39 (d, J=5.85) |
| 1-H | 3.90 (d, | 3.82 (d, J=4.96) | 3.87 (d, J=5.16) |
| | J_{11,12}=5.13) | | |
| 12-H | 4.26 (dd, J=5.13, | 4.11 (d, J=4.1) | 4.30 (d, J=4.41) |
| | 1.46) | | |
| 14-H | 3.12 (d, J=1.46) | 3.06 (br) | 2.97 (d, J=8.29) |
| | 2.56 (sept) | 2.56 (m) | 2.34 (m) |
| 15-H | | | |
| 16-H₃ | 0.89 (d, J=6.97) | 0.85 (d, J=6.93) | 0.79 (d, J=6.83) |
| 17-H₃ | 1.00 (d, J=6.6) | 0.94 (d, J=6.94) | 0.91 (d, J=6.81) |
| 19-H₂ | 4.74 (m) | 4.62 (br) | 4.89 (d, J=17.5) |
| | | | 4.78 (d, J=17.5) |
| 20-H₃ | 1.12 (s) | 1.07 (s) | 0.96 (s) |
| 13OH | | | 4.75 (s) |
| 14OH | | | 3.67 (d, J=8.3) |

*All chemical Shifts are given in ppm and coupling constants in Hz

TABLE 2
¹³C-NMR Spectral Data of T4*

| | T4 (DMSO-d₆, | |
|---|---|---|
| C | Literature)³ | T4 (DMSO-d₆) |
| 1 | 30.41t | 30.19t |
| 2 | 17.01t | 17.01t |
| 3 | 125.27s | 123.56s |
| 4 | 161.20s | 162.60s |
| 5 | 39.88d | 39.07d |
| 6 | 23.26t | 22.77t |
| 7 | 61.31d | 61.33d |
| 8 | 60.50s | 60.90s |
| 9 | 70.02s | 68.65s |
| 10 | 35.74s | 35.51s |
| 11 | 57.66d | 58.50d |
| 12 | 59.08d | 58.36d |
| 13 | 76.58s | 76.10s |
| 14 | 76.24d | 75.45d |
| 15 | 28.98d | 29.11d |
| 16 | 15.17q | 15.83q |
| 17 | 15.38q | 15.94q |
| 18 | 174.19s | 173.51s |
| 19 | 70.74t | 70.65t |
| 20 | 13.27q | 14.18q |

*All chemical Shifts are given in ppm and coupling constants in Hz

TABLE 3
¹H-NMR Spectral Data of T11*

| | T11 (CDCl₃, | |
|---|---|---|
| Proton | Literature)³ | T11 (DMSO-d₆) |
| 1-H₂ | 1.25, 1.38 | 1.39 (dd, J=12.50, |
| | | 4.62) |
| | | 1.25 (m) |
| 2-H₂ | 2.13 (m), 1.97 (m) | 2.13 (m), 1.98 (m) |
| 5-H | 2.65 (br, d) | 2.68 (br, d) |
| 6-H_{α} | 2.17 (m) | 2.18 (m) |
| 6H_{β} | 1.87 (t, J=11) | 1.84 (t, J=13.78) |
| 7-H | 3.32 (d, J=6) | 3.31 (d, J=7.11) |
| 11-H | 3.69 (d, J=5) | 3.69 (d, J=5.42) |
| 12-H | 3.82 (d, J=5) | 3.82 (m) |
| 14-H | 2.93 (s) | 2.94 (dd, J=7.39, |
| | | 1.54) |
| 15-H | 2.26 (sept, J=7) | 2.27 (m) |
| 16-H₃ | 0.76 (d, J=7) | 0.77 (d, J=6.90) |
| 17-H₃ | 0.88 (d, J=7) | 0.88 (d, J=6.83) |
| 19-H₂ | 4.84 (q) | 4.88 (d, J=18.76) |
| | | 4.78 (d, J=18.37) |
| 20-H₃ | 0.95 (s) | 0.95 (s) |
| 12OH | | 4.30 (d, J=9.21) |
| 13OH | | 4.26 (s) |
| 14OH | | 4.53 (d, J=7.38) |

*All chemical Shifts are given in ppm and coupling constants in Hz

TABLE 4
¹C-NMR Spectral Data of T11*

| | T11 (CD₃OD | |
|---|---|---|
| C | Literature)³ | T11 (DMSO-d₆) |
| 1 | 32.21t | 30.65t |
| 2 | 18.76t | 17.56t |
| 3 | 126.32s | 124.09s |
| 4 | 165.10s | 163.39s |
| 5 | 41.86d | 41.23d |
| 6 | 24.86t | 23.36t |
| 7 | 63.51d | 61.49d |
| 8 | 63.23s | 61.99s |
| 9 | 67.93s | 66.25s |
| 10 | 37.32s | 35.73s |
| 11 | 60.59d | 59.23d |
| 12 | 70.51d | 68.72d |
| 13 | 77.15s | 75.19s |
| 14 | 78.41d | 76.74d |
| 15 | 29.60d | 28.07d |
| 16 | 16.58q | 16.38q |
| 17 | 16.79q | 16.55q |
| 18 | 177.01s | 174.08s |
| 19 | 72.92t | 71.18t |
| 20 | 15.25q | 14.82q |

*All chemical Shifts are given in ppm and coupling constants in Hz

TABLE 5
¹H-NMR Spectral Data of T8*

| | T8 (CDCl₃, | |
|---|---|---|
| Proton | Literature)⁴ | T8 (DMSO-d₆) |
| 1-H₂ | | 1.33 (m) |
| 2-H₂ | 4.71 (m) | 4.33 (br) |
| 5-H | | 2.50 (m) |
| 6-H_{α} | | 2.22 (m) |
| 6H_{β} | | 1.93 (t, |
| | | J=14.00) |
| 7-H | | 3.36 (d, |
| | | J=5.51) |
| 11-H | | 3.89 (d, |
| | | J=2.81) |
| 12-H | | 3.54 (d, |
| | | J=2.50) |
| 14-H | | 3.32 (d, |
| | | J=9.87) |
| 15-H | | 2.15 (m) |
| 16-H₃ | | 0.79 (d, |
| | | J=6.83) |
| 17-H₃ | | 0.91 (d, |
| | | J=6.81) |
| 19-H₂ | 4.86 (t, J=1.5) | 4.85 (br) |
| 20-H₃ | 1.41 (s) | 0.96 (s) |
| 2OH | 1.76 (bs) | 5.10 (d, |
| | | J=4.92) |
| 14OH | | 4.63 (d, |
| | | J=7.64) |

*All chemical Shifts are given in ppm and coupling constants in Hz

TABLE 6
¹³C-NMR Spectral Data of T8 and L2

| | L2 (DMSO-d₆, | |
|---|---|---|
| C | Literature)³ | T8 (DMSO-d₆) |
| 1 | 28.96t | 39.07t |
| 2 | 16.55t | 58.72d |
| 3 | 123.02s | 125.52s |
| 4 | 162.32s | 164.50s |
| 5 | 39.93d | 40.73d |
| 6 | 22.60t | 22.63t |
| 7 | 59.70d | 60.22d |
| 8 | 60.73s | 61.32s |
| 9 | 64.34s | 64.84s |
| 10 | 35.17s | 35.48s |
| 11 | 55.23d | 56.24d |
| 12 | 54.46d | 54.53d |
| 13 | 62.90s | 64.96s |
| 14 | 71.56d | 71.63d |
| 15 | 35.72d | 27.80d |
| 16 | 61.59t | 17.02q |
| 17 | 12.73q | 17.96q |
| 18 | 173.05s | 172.73s |
| 19 | 70.12t | 70.03t |
| 20 | 13.64q | 15.96q |

*All chemical Shifts are given in ppm and coupling constants in Hz

The molecular weights of T4, T8 and T11 are shown in Table 7.

TABLE 7
The Molecular Weight of T4 T8 and T11

| Sample Name | Sample Code | Molecular Formula | Molecular Weight |
|---|---|---|---|
| Tripchlorolide | T4 | C₂₀H₂₆O₆Cl | 396 |
| Tripdiolide | T8 | C₂₀H₂₄O₇ | 376 |
| Triptriolide | T11 | C₂₀H₂₆O₇ | 378 |

Quantitation of the Compounds T4, T8 & T11

Table 8 shows the amounts and purity of each of these three compounds purified from about 253 g of AHT-323A botanic extract. About 174 mg of T4, 48 mg of T8 and 237 mg of T11 were obtained from about 253 g of the extract. Thus, the net weights of pure T4, T8 and T11 determined to be 166.3 mg, 47.3mg, and 233mg, respectively, by weighing the purified substances. Based on these determinations, the amounts of T4, T8 and T11 in 253 g of the AHT-323A botanical extract are determined to be 0.657 g/mg (about 0.06% by weight), 0.187 g/mg (about 0.018% by weight), and 0.921 g/mg (about 0.09% by weight), respectively. When a 10% loss of the components at each step during the purification process is considered, the final concentrations of T4, T8 and T11 in the AHT-323A botanical extract is determined to be 1.237 g/mg (about 0.013% by weight), 0.352,g/mg (about 0.036% by weight), and 1.733.g/mg (0.18% by weight), respectively. This was calculated based on the following: a total of 253 g crude botanical extract was used as a starting material for the purification. Six steps in total was used for the purification of each of the single compounds above. Each step was estimated to loss approximately 10% of yield for each of the compounds. The molar ratio for T11:T4:T8 was estimated to be 0.5-1.0:0.4-0.7:0.1-0.2.

Any other methods of quantifying T4, T8 and T11, which is apparent to a person of ordinary skill in the art can also be used in lieu of the above described methods to achieve substantially the same results.

TABLE 8
The Isolation Amount and Purity of T4 T8 and T11

| | | | | Final |
|---|---|---|---|---|
| | Gross weight | Purity | Net weight | concentration |
| Sample name | (mg) | (%) | (mg)^{▴} | (g/mg)* |
| Tripchlorolide T4 | 174 | 95.6% | 166.3 | 0.657 |
| Tripdiolide T8 | 48 | 98.6% | 47.3 | 0.187 |
| Triptriolide T11 | 237 | 98.3% | 233.0 | 0.921 |

^{▴}final net weight = (gross weight) × purity;
*the crude botanical extract is 253 g

Six steps in total were used for the purification of each of the single compounds above. Each step was estimated to lose approximately 10% of the yield for each of the compounds. Therefore, the original weight of T11, T4 and T8 can be worked out according to the formula: x·(1-10%)⁶=final net weight. (x represents the original weight of T11, T4 or T8). The detail information can been seen in the following table:

| | | Estimate | | |
|---|---|---|---|---|
| Sample | Final net | of | Original | Original |
| name | weight (mg) | Loss | net weight (mg)^{▴} | content (g/mg)* |
| T11 | 233 | (1-10%)⁶ | 438.4 | 1.733 |
| T4 | 166.3 | | 313.0 | 1.237 |
| T8 | 47.3 | | 89.0 | 0.352 |

^{▴}original net weight = (final net weight)/loss rate;
*the crude botanical extract is 253 g}

III. Determination of the Amount of T10 in AHT-323A Botanic Extract

Purification T10

Purified T10 compound was purchased from Fujian Provincial Research Institute of Medicine. The method for the purification of T10 as well as its chemical and physical properties was previously reported by Fuxiao Deng et al. in Fujian Pharmaceutical Journal, 1980,4: (2), the content of which is hereby incorporated by reference in its entirety. It can be purchased from Fujian Provincial Research Institute of Medicine, China.

Alternatively, T10 may be purified from AHT-323A botanical extract by further purification through a silica-gel chromatographic column eluted with chloroform. The Chloroform elute was again loaded on the silica-gel chromatographic column followed by elution with ethyl acetate-petroleum. The elute was recrystalized to yield purified T10. Chen Jun Yuan et al., Pharm aceutical Industry, 1989, (5):195 (the reference is hereby incorporated by reference in its entirety.)

Quantification of T10

The following description is an example for determining the amount of T10 in AHT-323A botanical extract directly without further purification of the T10. Other methods that is known to a person of ordinary skill in the art may also be employed to achieve a substantially the same result.

A reversed phase HPLC method for determination of the amount of T10 was implemented. The method utilizes a Waters Symmetry C 18 (4.6×250 mm, 5μ) column with UV detection at 218 nm. The mobile phase consists of acetonitrile and 0.2% v/v phosphoric acid.

Preferred HPLC Equipment, Reagents and Conditions:

Pump: Shimadzu LC 10ATVP; detector: Shimadzu SPD-M10AV; autosampler: Shimadzu SIL-10ADVP; software: Shimadzu LC 10, Version 1.63; column over: Shimadzu CTO-10AVP. T10 was obtained from Fujian Provincial Research Institute of Medicine; AHT-323A botanic extract was prepared as described above; acetonitrile: HPLC Grade, BDH; Nylon filter: 0.45μ, 30 mm diammeter (Bonner); ortho-Phosphoric acid, 85% reagent grade ACS (Scharlau); water: HPLC grade.

HPLC conditions: column: Water Symmetry, 4.6×250 mm, 5μ, C18; temperature: ambient; injection volume: 30 μL; wavelength: 218 nm; flow rate: 1.8 mL/min; mobile phase: A (0.2% v/v H₃PO₄), B (acetonitrile); sample solvent: methanol.

The mobile phase for the HPLC analysis is a gradient solution preferably comprising solution A and solution B as defined above, where solution B is applied to the HPLC column at a concentration of about 19% (The balance comprises solution A.) at the beginning followed by applying about 100% of solution B after about 28 minutes from the beginning, applying about 19% of solution B after about 34 minutes from the beginning, and applying 0% of solution B after about 45 minutes from the beginning, as shown below:

| | | |
|---|---|---|
| 0.01 min | solution B | 19.0% |
| 28.00 min | solution B | 19.0% |
| 28.50 min | solution B | 100.0% |
| 34.00 min | solution B | 100.0% |
| 34.50 min | solution B | 19.0% |
| 45.00 min | stop | |

25 mg of a AHT-323A botanic extract sample was weighed into 25 ml volumetric flask. 10 ml of methanol was added and the sample was sonicated for 10 minutes. The sample was then made up to volume with methanol to give a working sample concentration of 1 mg/ml.

A T10 standard was prepared by initially weighing 25 mg of T10 into 25 ml volumetric flask. Afterwards, 5 ml of methanol was added and the standard was dissolved by sonication. The standard was then made up to volume with methanol to give 1 mg/ml (stock standard concentration) and this is further diluted with methanol to give the final working standard concentration of 0.001 mg/ml. This concentration was derived on the basis of the limited of concentration allowed for T10 in the AHT-323A botanic extract (i.e. 10 g/10000 g).

A suitability test was conducted as an integral part of the analytical method and it ascertains the suitability and effectiveness of the operating system.

The analysis was conducted using external standard method. The working standard solution of T10 was injected onto the HPLC system. The average retention time of the T10 in standard solution for this system was found to be at 25.43 minutes.

The system suitability parameters observed were as shown in Table 9.

TABLE 9
System Suitability Parameters

| | Retention | | Theoretical | Area RSD (%) |
|---|---|---|---|---|
| Peak | Time (min) | Tailing | Plates | For 6 replicates |
| T10 | 25.37 | 0.95 | 12602 | 0.5 |

Acceptance criteria: Tailing factor should ≦ 1.5

The system precision-repeatability was performed to determine the degree of agreement between a series of measurement under the same conditions. The system precision demonstrates the performance and function of the HPLC system used for its intended purpose.

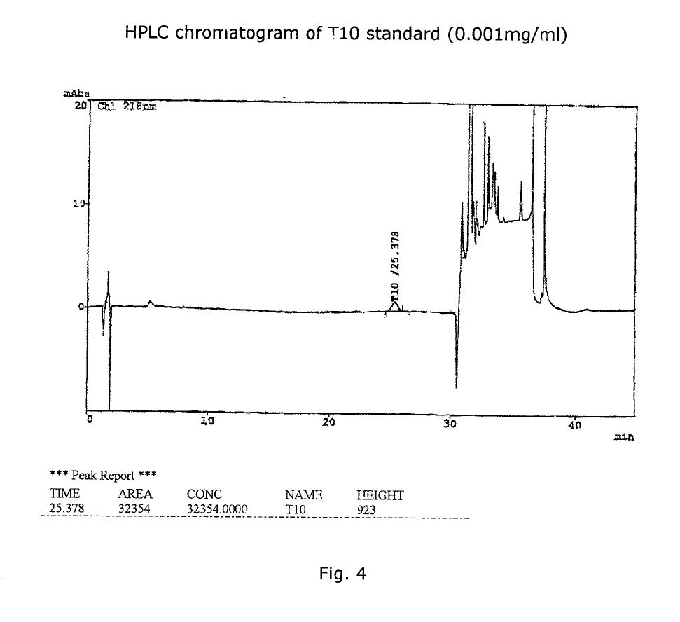

A working standard containing T10 at a concentration of 0.001 mg/ml was prepared and analyzed as shown in FIG. 4. Six injection responses were recorded. The results were as shown as in Table 10:

TABLE 10
Height Response and Retention Time for T10

| Replicate Number | Height Response | Retention Time |
|---|---|---|
| 1 | 972 | 25.37 |
| 2 | 925 | 25.58 |
| 3 | 923 | 25.38 |
| 4 | 925 | 25.24 |
| 5 | 1048 | 25.44 |
| 6 | 975 | 25.54 |
| Mean | 961 | 25.43 |
| % RSD | 5.1 | 0.5 |

Linearity is performed to determine the range in which an analytical method will obtain results that are directly proportional to the concentration of analyte in the sample. The linearity of detector response of T10 was determined at the concentration of 25%, 50%, 100%, 200% and 400% of the working standard concentration.

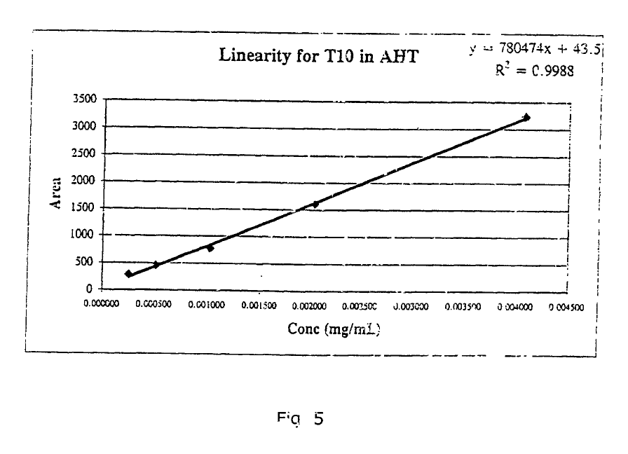

Triplicate injections were made at each concentration. The area responses were tted against the corresponding concentrations and a linear regression analysis was performed the resulting data as show in Table 11 and FIG. 5.

TABLE 11
Linearity for T10

| | Concen- | Equivalence to the | Observed | Response |
|---|---|---|---|---|
| Theoretical | tration | T10 in AHT-323A | Average | Concen- |
| Concentration | (mg/ml) | Extract (g/g) | Height | tration |
| 25% | 0.000254 | 2.5/10000 | 285 | 1119853.1 |
| 50% | 0.000508 | 5/10000 | 459 | 903488.1 |
| 100% | 0.001017 | 10/10000 | 776 | 763506.4 |
| 200% | 0.002034 | 20/10000 | 1609 | 791371.6 |
| 400% | 0.004067 | 40/10000 | 3239 | 796371.0 |

| | |
|---|---|
| For concentration response: mean: | 874918.0 |
| % RSD: | 16.37 |
| Coefficient of determination (R²) | 0.9988 |
| Slope | 780474 |
| Y-intercept | 43.5 |

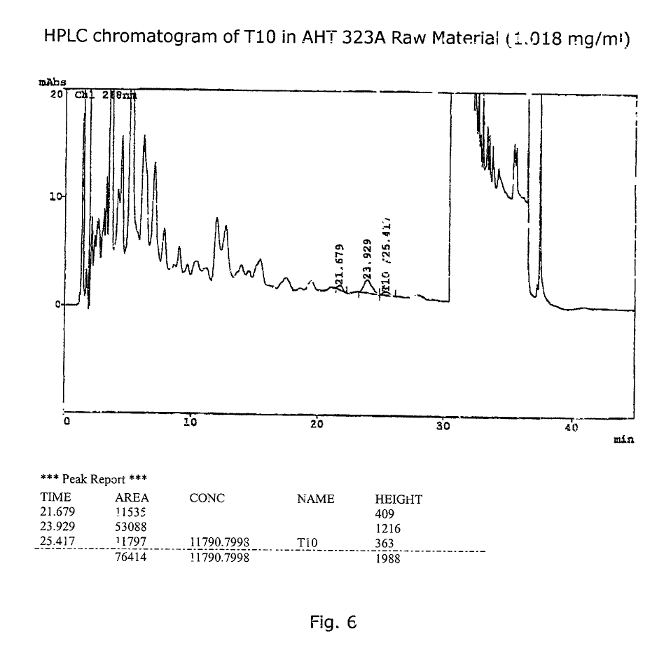

The accuracy of the quantitative HPLC analysis was determined by spiking AHT-323A botanic extract sample (at the working sample concentration of 1 mg/ml) with T10 at levels representing 80%, 100% and 120% of the working standard concentration, as shown in FIG. 6. The mean recovery observed was 95.3%.

An AHT-323A botanical extract was further analyzed by the HPLC method. The T10 peak can be identifiably separated from the other peaks in the extract with a resolution value of 1.56 using the HPLC gradient method. Duplicates of AHT-323A botanic extract samples were prepared at a concentration of 1 mg/ml. The concentration of T10 in AHT-323A botanic extract was determined to be 4 g/10000 g (0.04%).

IV. The Fingerprinting Technology for AHT-323 Botanical Extract

The fingerprinting technology based on gradient HPLC methodology were employed to create a unique chromatographic profile of AHT-323A botanical extract, where all identifiable and characteristic components in the present botanic extract were captured as a “fingerprint”, which provides characteristic information of the present botanical extract.

The fingerprint chromatography was introduced and subsequently accepted by WHO for quality control of herbal medicines. The availability of on-line diode array detection in the fingerprint technology offers an accurate picture of herbal medicines.

The purified T11 T4 T8 and T10 were used as analytical markers to determine the quantities and retention times of these four components in AHT-323A botanical extract. In order to calculate the relative retention time of the peaks of interest, suitable internal references was tested and hydrocortisone was selected as it shares similar molecular structure to the above mentioned four components:

Preferred HPLC Equipment, Reagents and Conditions:

Pump: Shimadzu LC 10ATVP; detector: Shimadzu SPD-M10AV with photo diode array; autosampler: Shimadzu SIL-10ADVP; software: Shimadzu LC 10, Version 1.63; column oven: Shimadzu CTO-10AVP. AHT-323A botanic extract was prepared as described above; purified T4, T8, and T11 were prepared as described above; purified T10 was obtained from Fujian Provincial Research Institute of Medicine; acetonitrile: HPLC Grade, BDH; Nylon filter: 0.45μ, 30 mm diameter (Bonner); ortho-Phosphoric acid, 85% reagent grade ACS (Scharlau); water: HPLC grade; Hydrocortisone: USP (Sigma); Methanol: HPLC grade, BDH.

HPLC conditions: column: Water Symmetry, 4.6×250 mm, 5μ, C18; temperature: ambient; injection volume: 40 μL; wavelength: 214 nm; flow rate: 2.0 mL/min; run time: 161 minutes; mobile phase: solution A (acetonitrile), solution B (0.2% v/v H₃PO₄); sample solvent: methanol : 0.2% v/v H₃PO₄: acetonitrile:HPLC grade water=65:10:5:20.

While a commercially available HPLC column such as the Water Symmetry C18 column is preferred, a HPLC column may be prepared by using an octadecyl silane chemically bonded to porous silica or ceramic micro-particles having a 3 to 10 um diameter, or an octadecyl silane chemically bonded to silica gel of a controlled surface porosity that has been bonded to a solid spherical core having a 30 to 50 um diameter, or porous silica particles having a 5 to 10 um diameter, or silica gel having controlled surface porosity and bonded to a solid spherical core having a 30 to 50 um diameter, or an octylsilane chemically bonded to totally porous silica particles having a 3 to 10 um in diameter, or nitrile groups chemically bonded to porous silica particles having a 3 to 10 um diameter, or phenyl groups chemically bonded to porous silica particles having a 5 to 10 um diameter, or a hexylsilane chemically bonded to porous silica particles having a 3 to 10 um diameter, or a dimethylsilane chemically bonded to porous silica particles having a 50 to 10 um diameter, or dihydroxypropane groups chemically bonded to porous silica particles having a 5 to 10 um in diameter, or the like.

A gradient method was employed for the HPLC analysis. The mobile phase of the gradient solution comprises ortho-phosphoric acid, methanol, acetonitrile or a mixture thereof. Preferably, the gradient solution comprises solutions A and B as described above, where solution B is applied to the HPLC column at a concentration of about 93% (the balance comprises solution A) at the beginning followed by applying about 80% of solution B after about 120 minutes from the beginning, applying about 70% of solution B after about 160.00 from the beginning, and applying 0% of solution B after about 161 minutes from the beginning, as shown below:

| | | |
|---|---|---|
| 0.01 min | solution B | 93% |
| 120.00 min | solution B | 80% |
| 160.00 min | solution B | 70% |
| 161.00 min | STOP | 0 |

Preparation of the Standards:

Four standards, i.e. purified T11, T4, T8 and T10, were prepared for the purpose of the qualitative experiment in order to determine their retention time under the preferred HPLC conditions stated above. The four standards were prepared by diluting 0.1 ml of each stock standard at the concentration of 1 mg/ml in 5 ml methanol to obtain a final concentration of 0.02 mg/ml respectively. 0.02 mg/ml is the working concentration for each of T11, T4, T8 and T10.

Preparation and Testing of AHT-323A Botanical Extract:

50 mg of AHT-323A botanical extract was weighed into a 25 ml volumetric flask. 5 ml of water was added to the flask and sonicated for 10 minutes. 5 ml of methanol was added and the botanic extract sample was sonicated for a further 10 minutes until fully dissolved. The raw material solution was shaken for 10 minutes at 600 oscillations per minute. After 10 minutes, 2.5 ml of 0.2% H₃PO₄, 1 ml of acetonitrile and 5 ml of methanol were added and the solution was sonicated for another 10 minutes followed by another 10 minutes of shaking. The solution was cooled and was made to volume with methanol.

3.84 mg of hydrocortisone was weighed into a 50 ml volumetric flask. 10 ml of methanol was added to the flask and was sonicated for 10 minutes. The flasks were left to cool and solution was made up to volume with methanol. The solution was cooled and was made to volume with methanol.

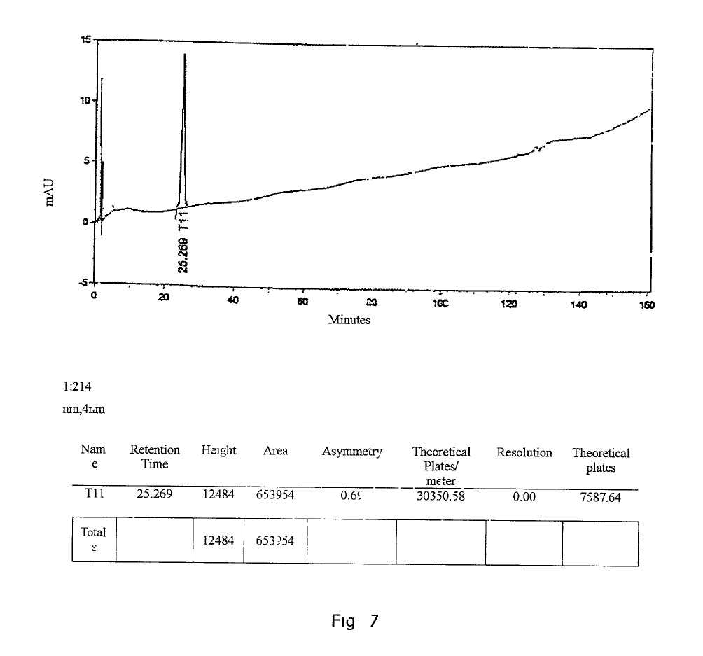

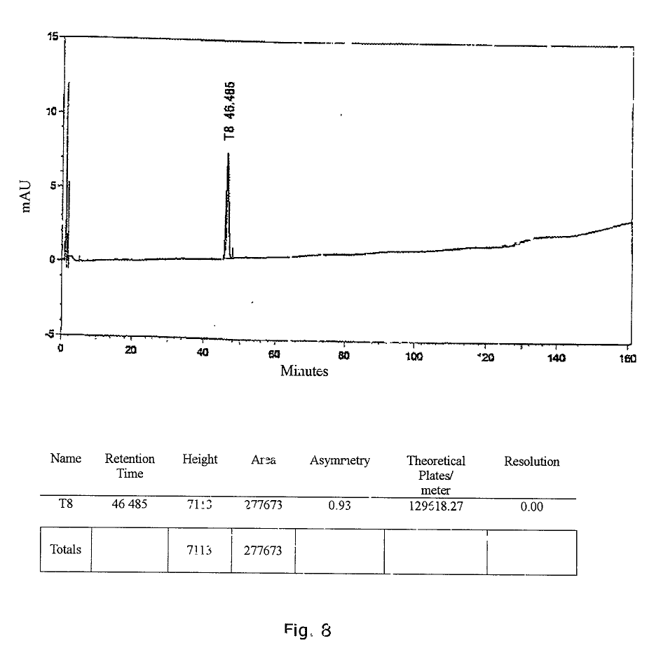

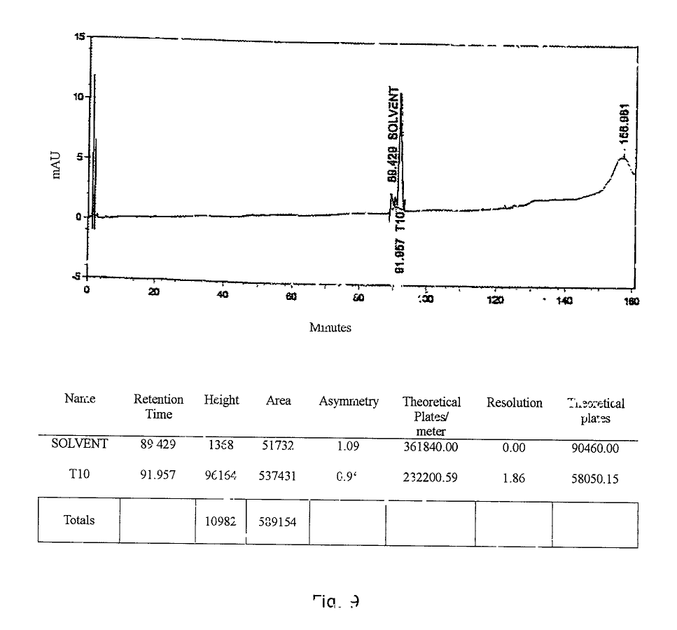

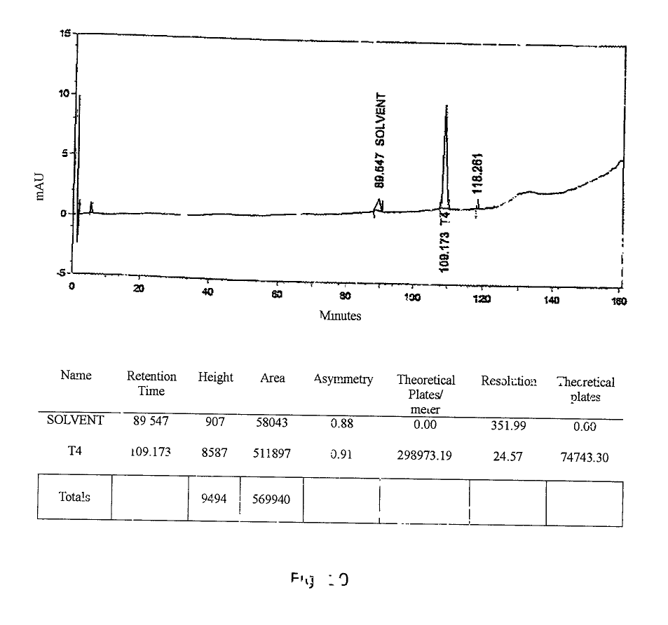

An instrument suitability test is an integral part of the analytical method and it ascertains the suitability and effectiveness of the operating system. The test was conducted using external standard method. The working standard solution of each individual marker and Hydrocortisone followed by a mix of all the four purified markers T11, T4, T8 and T10, was injected onto the HPLC system. The average retention times of the standards and Hydrocortisone in standard solutions for this system were found to be:

| | Retention time | Rf value (see Appendix N) |
|---|---|---|
| Triptriolide (T11, FIG. 7) | 26.3 | 0.19 |
| Tripdiolide (T8, FIG. 8) | 46.5 | 0.30 |
| Triptolide (T10, FIG. 9) | 92.0 | 0.76 |
| Tripchloride (T4, FIG. 10) | 109.7 | 0.48 |
| Hydrocortisone | 115.6 | |

The retention time of each of the peaks of T11, T4, T8 and T10 is comparable to the Rf values. T11 is the most polar compound, hence lowest Rf, as it has 3 hydroxyl groups and was eluted first from he reverse phase column. T8 has two hydroxyl groups therefore it is less polar than T11. Even though T4 has a reported lower Rf value than T10, the former is eluted last from the reverse phase column.

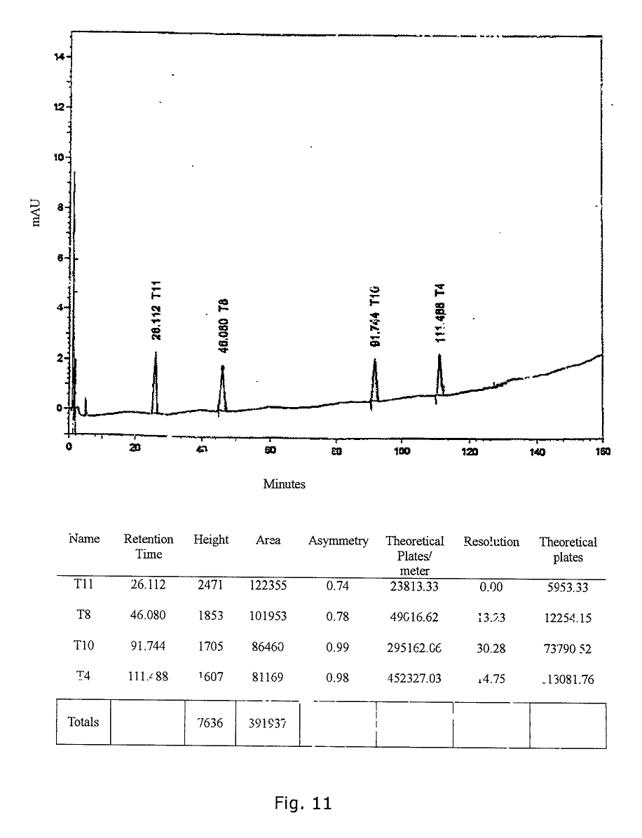

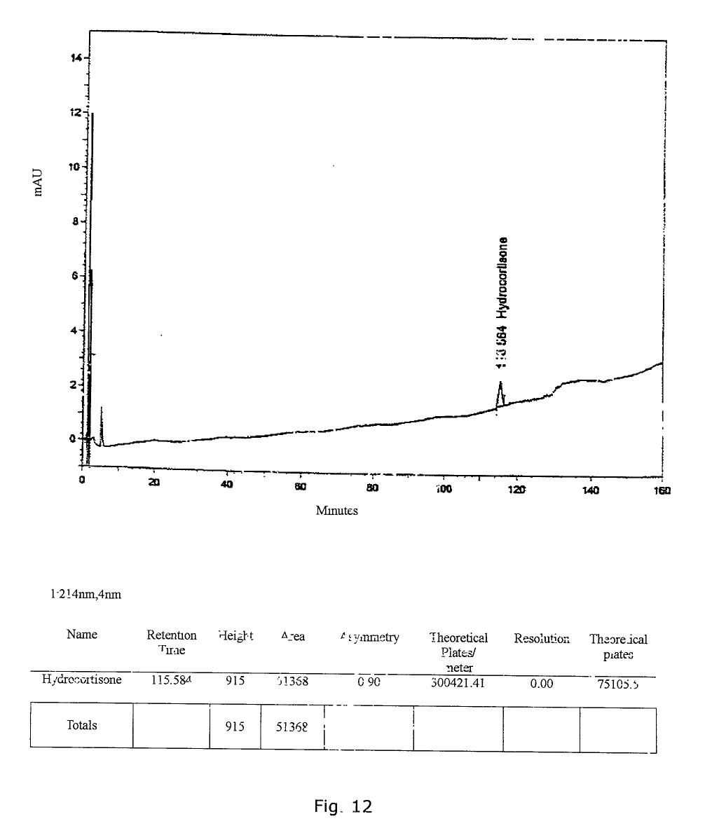

Three steroids, hydrocortisone, cortisone and prednisone were individually tested and their retention times were compared with the retention time of the four markers. When hydrocortisone was injected during the run using the present gradient method it was well resolved from T4 and hence will be employed as the internal reference of choice. See FIGS. 11 and 12 and also Table 12.

TABLE 12
Retention Times of Markers and Hydrocortisone
under HPLC gradient Method

| Peak | Retention time (min) | RRT |
|---|---|---|
| T4 | 111.5 | 0.97 |
| T8 | 46.1 | 0.40 |
| T10 | 91.7 | 0.80 |
| T11 | 26.1 | 0.23 |
| Hydrocortisone | 114.1 | 1.00 |

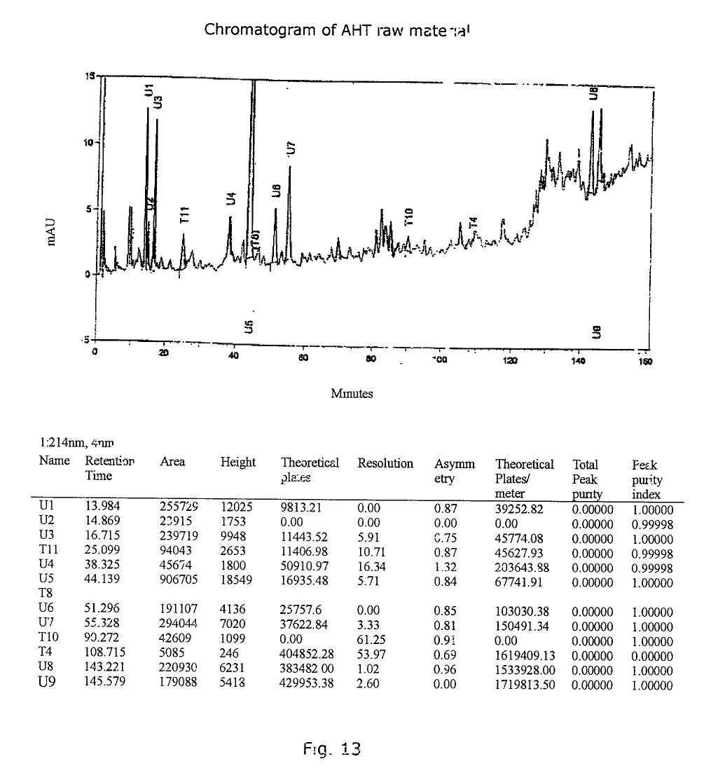

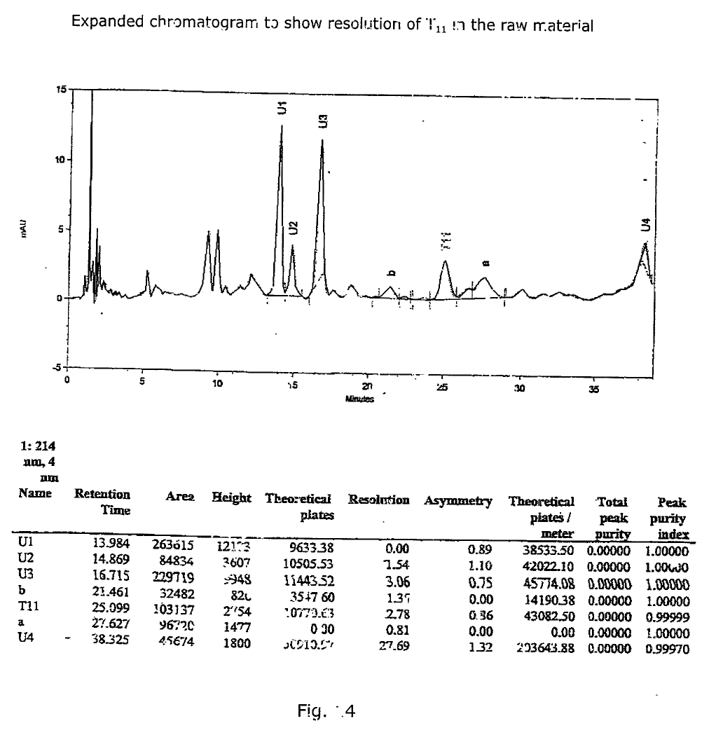

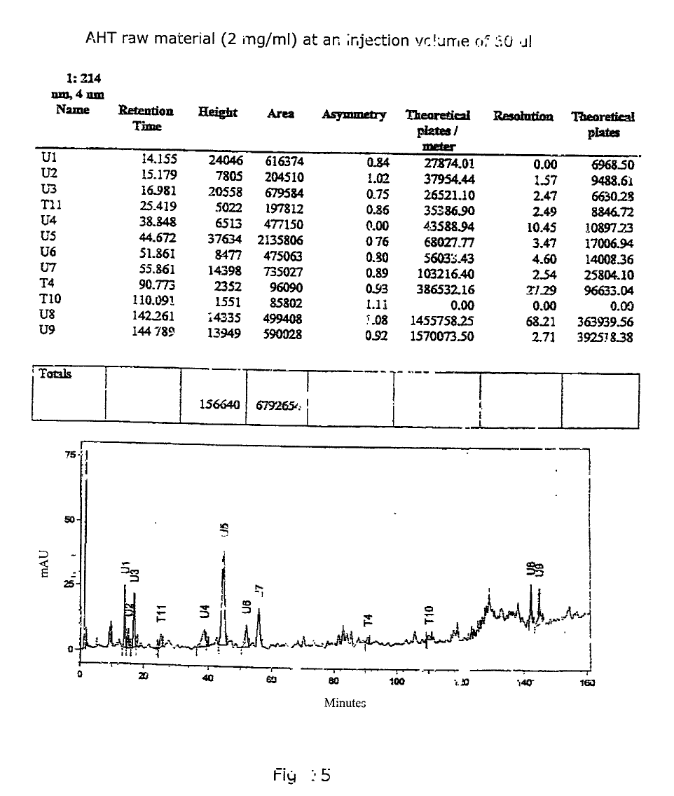

The AHT-323A botanic extract was then tested. 40 ul of the solutions of AHT-323A botanic extract having a concentration of 1 mg/ml was injected, see FIG. 13. It was found that the T11 peak is large enough for quantification. Peak T11 is well resolved from all surrounding peaks, see FIG. 14. Peaks of T8, T10, and T4 are resolved for other peaks in the sample, however, they are hardly quantifiable due to the small peak area. When the purity index of T11 was calculated, it was found to be 1, a good indicator that T11 was resolved and no other ingredients were found to have the same retention time as T11 as shown in FIG. 15.

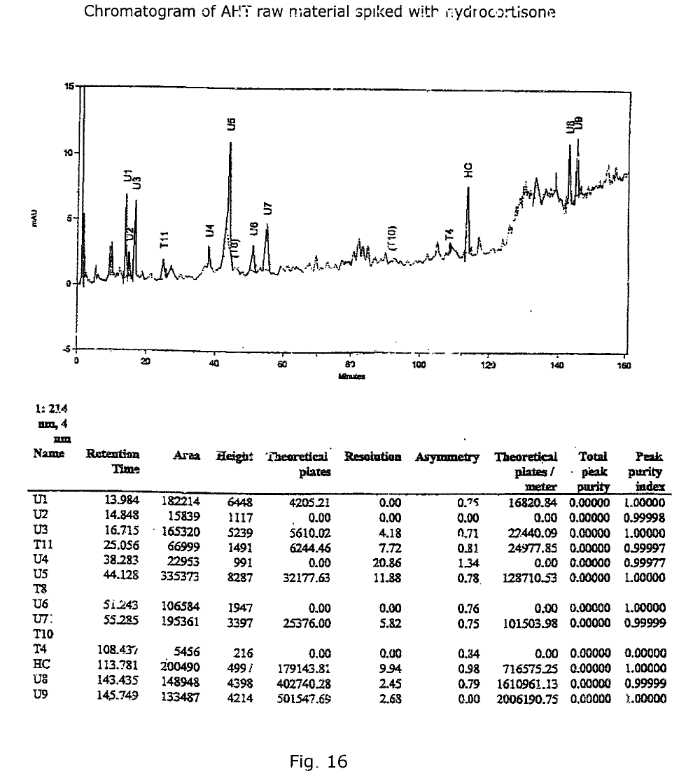

The AHT-323A botanical extract sample is then injected with hydrocortisone. 1 ml of hydrocortisone stock solution was added to a 5 ml volumetric flask and was made to volume with the 1 mg/ml solution of the AHT-323A botanical extract sample. Then 40 μl of this solution was injected resulting FIG. 16.

Calculation of RRT Against Hydrocortisone

The chromatograms of the AHT-323A botanical extract gives a unique HPLC profile. Apart from the four individual markers found in these samples, there are several well resolved and distinct peaks which can be employed as the unique fingerprinting indicators for this product. Peaks which are marked with “U” were selected as unknown peaks for fingerprinting and their relative retention times (RRT) were calculated against hydrocortisone, as shown in Table 13.

TABLE 13
Retention Time of Peaks and Calculated RRT against Hydrocortisone

| | Retention time | | |
|---|---|---|---|
| Peak Name | (min) | RRT | RSD (%) for RRT |
| U1 | 14.0 | 0.12 | 0.26 |
| U2 | 14.9 | 0.13 | 0.49 |
| U3 | 16.8 | 0.15 | 0.33 |
| U4 | 38.4 | 0.34 | 0.24 |
| U5 | 44.2 | 0.39 | 0.25 |
| U6 | 51.4 | 0.45 | 0.17 |
| U7 | 55.4 | 0.49 | 0.19 |
| U8 | 143.5 | 1.26 | 0.28 |
| U9 | 145.8 | 1.28 | 0.24 |
| Hydrocortisone | 114.1 | 1.00 | n/a |
| T11 | 25.1 | 0.22 | 0.26 |

The RRT of each peak were taken from an average of four individual runs. The retention time of the hydrocortisone is an average of this peak from three runs. The RRT is defined as a retention time (min.) of a peak/the retention time (min.) of a internal reference (hydrocortisone).

The fingerprint analysis is especially useful in quality control and stability tests. It can be shown that T11, T8, T4, and T10 are consistently present in the AHT-323A botanical extract from different batches. According to EEC guideline 75/318 “quality of Herbal Drugs”, the fingerprint analysis allows us to identify the very typical compounds in the herbal extract and finished products.

The AHT-323A botanical extract produces a large number of peaks in HPLC chromatograms within the 161 minutes run with four known peaks T11, T8, T4, and T10 and nine unknown peaks being assigned. The current HPLC method is successful in separating and detecting all peaks of interest in the extract. Hydrocortisone is selected as an internal reference for calculation of relative retention time for the peaks of interest for the purpose of fingerprinting identification. The characteristic chromatographic profiles for the AHT-323A botanical extract can be reproduced by this fingerprinting technology. The assignment of these peaks can also be done form the on line UV spectra and elution profile.

V. Determination of the Amount of T11 in AHT-323A Botanical Extract by HPLC Methodology

The equipment and reagents describe below are preferred to practice the present invention but will not limit the scope of the present invention in any way. They are commercially available and can be readily obtained. It is apparent to a person of ordinary skill in the art that other equipment and reagents may perform substantially the same function in substantially the same way to achieve substantially the same result.

Equipment: HPLC with UV-Vis Photodiode Array detector, gradient pump and auto injector with suitable data processor; analytical balance; ultrasonic bath; 0.45 micron nylon filters; mechanical shaker; HPLC syringe.

Reagents: methanol (HPLC grade); acetonitrile (HPLC grade); water (HPLC grade); ortho-phosphoric acid (AR Grade); sample Solvent: Prepare 0.2% v/v solution of ortho-phosphoric acid in water.

Method: Determination of the amount T11 in AHT-323A Botanial Extract was performed using gradient HPLC methodology and having purified T11, T4, T8 and T10 as standards and hydrocortisone (USP) as internal reference. The preferred experimental conditions are described below:

Chromatographic System/Conditions

| | | |
|---|---|---|
| Column: | Type: | Waters Symmetry C18 |
| | Dimensions: | 4.6 × 250 mm |
| | Particle Size: | 5 microns |
| Detector: | Type: | UV-Vis Photodiode Array Detector |
| | Wavelength: | 214 nm |
| | Sensitivity: | (May vary depending on detector used) |

| | |
|---|---|
| Flow Rate: | 2.0 mL/min |
| Column Temp: | Ambient |
| Total run time: | 170 minutes |
| Number of injections per | 2 |
| sample: | |
| Injection volume: | 50 μl |

Mobile Phase

Solution A: Acetonitrile

Solution B: 0.2% v/v ortho-phosphoric acid in water

De-aerate each mobile phase and run from two different flow channels

The mobile phase of the HPLC is a gradient solution preferably comprising solution A and solution B which are defined above, where solution B is applied to said HPLC column at a concentration of about 93% (the balance comprises solution A) at the beginning followed by applying about 80% of solution B after about 120 minutes from the beginning, applying about 70% of solution B after about 160 minutes from the beginning, applying about 93% of solution B after about 161 minutes, and applying 0% of solution B after about 170 minutes from the beginning as shown below:

Gradient Flow Program for HPLC

| | | | | | |
|---|---|---|---|---|---|
| 0.01 | min | Solution A | 7% | Solution B | 93% |
| 120.00 | min | Solution A | 20% | Solution B | 80% |
| 160.00 | min | Solution A | 30% | Solution B | 70% |
| 161.00 | min | Solution A | 7% | Solution B | 93% |
| 170.00 | min | STOP | | | |

Data Processing

Quantification by peak area, external standard method.

Internal Reference Preparation (Stock IR)

Weigh out accurately about 5.00 mg of Hydrocortisone SRS, transfer into a 50 mL volumetric flask. Dissolve in 10 ml methanol and sonicate for 10 minutes, allow to cool and dilute to volume with sample solvent, mix.

Standard Preparation (Prepare Only One Standard)

Step 1: Weigh out accurately about 5.00 mg of Triptiolode (T11), transfer into a 5 mL volumetric flask. Dissolve in methanol and sonicate for 10 minutes, allow to cool and dilute to volume with methanol, mix. (Stock solution).

Step 2: Further dilute 1 mL of the stock solution from step 1 to 10 ml with sample solvent.

Step 3: Further dilute 2.5 ml of solution form step 2 to 25 ml with sample solvent.

Step 4: Pipette 5 ml of the solution form step 3 and 2 ml of Stock IR into a 10 ml volumetric flask and dilute to volume with sample solvent. Use this as the working standard solution.

Step 5: Filter a portion of the working standard solution through 0.45 micron nylon filter into auto sampler vials, discarding the first 2 ml of filtrate.

System Suitability Preparation

Step 1: Prepare separate stock solutions containing 1 mg/ml of each of T4, T8 and T10 in methanol. Store this in the freezer and use it to prepare the system suitability standard.

Step 2: Place 50 μL(using syringe) of each of the stock solutions from Step 1 and the stock solution from Step 1 of the above “Standard Preparation” section into a 5 ml volumetric flask.

Step 3: Add 1 ml of the stock IR solution to the flask in Step 2 and dilute to volume with sample solvent. Mix well.

Step 4: Filter a portion of the solution from Step 3 through 0.45 micron nylon filter into auto sample vials, discarding the first 2 ml of filtrate.

System Suitability Test

Inject 50 μL of sample solvent to check baseline noise and drift or any other sign of system instability. Repeat injections if necessary until the system is stable. Note the retention time of any solvent related peaks.

Inject 50 μL of system suitability standard, check baseline noise and drift or any other sign of system instability. Repeat injections if necessary until the system is stable. Perform a column performance report and check the following.

Compare RT found to theoretical RT. Theoretical value is as follows:

| Compound | Approx. RT (min) |
|---|---|
| T11 | 24 |
| T4 | 107 |
| T8 | 43 |
| T10 | 87 |
| Hydrocortisone | 114 |

Take appropriate action if the T11 and Hydrocortisone drifts by more than 10% to the theoretical.

Calibration

Consecutively inject five 50 μL portions of working standard.

Calculate mean of the peak areas obtained for T11 in the working standard.

Take appropriate action if the R.S.D. of peak areas of T11 is greater than 2%.

Sample preparation (Spl) (Perform in Duplicate)

Step 1: Weigh accurately about 50 mg of the sample into a 25 ml volumetric flask and add 5 ml of methanol.

Step 2: Sonicate for 10 minutes or until dissolved, followed by 20 minutes of shaking at 600 oscillations per minute.

Step 3: Further add 10 ml of sample solvent and sonicate for another 10 minutes, allow to cool to room temperature.

Step 4: Add 5 ml of the stock IR solution to the volumetric flask with the sample in step 3 and dilute to volume with sample solvent and mix well. Shake for further 20 minutes, followed by sonication for 10 minutes. Cool and dilute to volume with sample solvent.

Step 5: Leave the solution to stand for 5 minutes for the precipitate to settle. Filter a portion through a 0.45 micron nylon filter into an autosampler vial discarding the first 2 ml of filtrate.

Step 6: Inject two 50 μL portion of each of the samples.

Calculations:

Step 1: Calculate the potency for each sample using the mean of the area:

T11(% w/w)=A Spl×mg std×P×spl dilution/A Std×std dilution×spl.wt

A spl=mean area of sample peak

A std=mean area of standard peak

P=purity of standard T11

Step 2: Take appropriate action if the difference between duplicate samples is greater than 10%.

Step 3: After every four samples it is normal to inject five additional injections of standard. For calculations on such groups it is normal practice to use the mean from the group of injections of standard made immediately before the four samples.

Step 4: If the relative standard deviation calculated on all the standard injections in the analysis run does not exceed 2% then the mean of all the standard responses may be used to calculate the results.

Step 5: If the difference between the group of injections of standard calculated at the beginning and the group of injections of standard calculated at the end of a group of samples exceeds 3% but is less than 5%, for T11 then the mean of all the standard responses may be used to calculate the results. If the difference is greater than 5% then that group of samples must be repeated.

The amount of T11 in AHT-323A botanical extract was determined according to the HPLC method described above is about 0.4%? (Do you have a HPLC profile for T11 quantification? I need a specific number for T11!)

The following example serves to further illustrate the present invention and not in any way to limit the scope of the present invention.

### EXAMPLE

Preparation of AHT-323A Botanic Extract

The plant roots of Tripterygium wilfordii Hook.F. (TW) harvested from Hunan Province of China were removed of foreign matter, stems and leaves. The roots were sun dried. The dried roots were visually examined by experienced professionals, tested using TLC methodology, and stored in a warehouse with good air flow. To prepare AHT-323A extract, the TW roots were transported from the warehouse to a pre-processing workshop, where the Radix TW was re-examined, manually removed of any contaminates (i.e., other botanical parts an foreign matter) and then was cut by machine into small pieces or ground to a powder.

The small pieces or the powder of Radix TW were then transported from the pre-processing workshop to an extraction workshop, loaded into a cyclic extractor, added with industrial grade ethanol and then extracted for 6 hours. The cyclic extraction process was then repeated five times.

The filtrates from each of the above ethanol extraction processes were collected, combined and then transmitted via tubes in an enclosed system to an ethanol recovery tank. The ethanol was then recovered at room temperature under reduced pressure. The ethanol extract was then transferred to a another extractor.

Chemical-grade chloroform was added to the ethanol extract at a volumetric ratio of 3:1 (chloroform to extract). Solution was mixed until completely dissolved, filtered and the filtrate collected in a storage tank. The remaining material was re-extracted and the filtrate added to the storage tank. This process was repeated until the filtered extract showed negative reaction to Kedde's Reagent by TLC. The chloroform was recovered and a dried crude powder extract was obtained by drying under heat (in water bath) at about 37° C. and under a reduced pressure of about 30±5 mm Hg.

The crude power from the chloroform extraction was then loaded (at a ratio of the crude extract to silica gel equaling to 1:10) onto large-scale production silica gel columns (6 m×22 cm stainless steel silica gel column) that have been equilibrated with chloroform. The columns were then eluted stepwise with gradient chloroform solutions containing 0.5-30% ethanol, respectively. The elution fractions were collected into cylindrical stainless steel containers.

The elution fractions underwent in-process testing for chemical constituents of diterpenoid compounds by TLC. The fractions showing positive for T4, T8 and T11 components were combined and concentrated via recovery of chloroform in water bath under a reduced pressure to obtain chloroform fluid extract. To remove chloroform residue, the fractions were dissolved in absolute ethanol, mixed and dried under heat (in water bath) and reduced pressure. This process can be repeated as needed.

The dried column extract was mixed, milled into fine powder in a Type V Blender, and screened through a 100-mesh vibrating sieve. Release tests were conducted on the fine powder using TLC and HPLC methodology. The final yield was estimated to be approximately 0.2% AHT-323A botanic extract, indicating that approximately 2.0 kg of the botanical extract was obtained from 1000 kg of dried Radix TW raw materials.

The following reference are cited to further illustrate or explain the present invention. The contents of these references are hereby incorporated by reference in their entirety.

References

(1) Chen, F. -X.; Lin, Y. -H.; Shi, M. -Y. In Xin Bian Zhong Cheng Yao Shou Ce; Zhong Guo Yi Yao Ke Ji Chu Ban She: Beijing, 1995; pp632.

(2) Zheng, J. K.; Fang, J. L.; Gu, K. X.; Xu, L. F.; Gao, J. W.; Sun, H. Z. ACTA Academiae Medicinae Sinicae 1987, 9, 323-328.

(3) Ma, P. C.; Lu, Z. Y.; Yang, J. J.; Zheng, Q. T. Acta Pharmaceutica Sinica 1991, 26, 759-763.

(4) Kupchan, S. M.; Court, W. A.; Dailey, R. G.; Gilmore, Jr.; C. J.; Bryan, R. F. J. Amer. Chem. S

(5) Chen Jun Yuan et al., Pharmaceutical Industry 1989, (5):195

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

We claim:

1. A botanical extract from the roots of plant Tripterygium Wilfordii Hook. F., comprising triptriolide (T11), tripchlorolide (T4), tripdiolide (T8) and triptolide (T10), wherein said T11 content is at least 0.09% by weight said T10 content is less than 0.05% by weight.

2. The botanical extract in claim 1, wherein the amounts of said T11 and said T10 are determined by a HPLC method.

3. The botanical extract in claim 1, wherein said T11 ranges from about 0.09% to about 0.4% by weight and said T10 ranges from 0.02% to about 0.05% by weight.

4. The botanical extract in claim 1, wherein the amounts of said T11 and said T10 are determined by a HPLC method.

5. The botanical extract in claim 1, wherein said T11 content is about 0.4% by weight and said T10 content is about 0.02% determined by a HPLC method.

6. The botanical extract in claim 1, wherein said T4 is at least 0.06% by weight.

7. The botanical extract in claim 6, wherein said T4 ranges from about 0.06% to about 0.13% by weight.

8. The botanical extract in claim 1, wherein said T8 is at least 0.018% by weight.

9. The botanical extract in claim 8, wherein said T8 ranges from about 0.018% to about 0.036% by weight.

10. The botanical extract in claim 1, wherein said T4 is at least 0.06% by weight and said T8 is at least 0.018% by weight.

11. The botanical extract in claim 10, wherein said T4 ranges from about 0.065% to about 0.13% by weight and said T8 ranges from about 0.018% to about 0.036% by weight.

12. A botanical extract from the roots of plant Tripterygium Wilfordii Hook. F., comprising triptriolide (T11), tripchlorolide (T4), and tripdiolide (T8), wherein said T11 content is at least 0.09% by weight, said T4 content is at least 0.06% by weight, and said T8 content is at least 0.018% by weight.

13. The botanical extract of claim 12, wherein said T11 ranges from about 0.09% to about 0.4% by weight, said T4 ranges from about 0.065% to about 0.13% by weight and said T8 ranges from about 0.018% to about 0.036% by weight.

14. The botanical extract of claim 13, wherein said T11 is about 0.4% by weight, said T4 is about 0.13% by weight and said T8 is about 0.036% by weight.

15. A botanical extract from the roots of plant Tripterygium Wilfordii Hook. F., comprising triptriolide (T11), tripchlorolide (T4), tripdiolide (T8) and triptolide (T10) having a molar ratio of T11:T4:T8:T10 equals to 0.5-1.0:0.4-0.7:0.1-0.2 :0.1-0.2.

16. A botanical extract from the roots of plant Tripterygium Wilfordii Hook. F. having a HPLC profile substantially the same as in FIG. 13.

17. A botanical extract from the roots of plant Tripterygium Wilfordii Hook. F. having a HPLC profile, said HPLC profile comprising peaks U1, U2, U3, U4, U5, U6, U7, U8, and U9, said U1, U2, U3, U4, U5, U6, U7, U8, and U9 peaks having respective retention times at about 14.0 minutes (U1), about 14.9 minutes (U2), about 16.8 minutes (U3), about 38.4 minutes (U4), about 44.2 minutes (U5), about 51.4 minutes (U6), about 55.4 minutes (U7), about 143.5 minutes (U8) and about 145.8 minutes (U9).

18. The botanical extract of claim 17, wherein said HPLC profile further comprises peaks of T11, T4, T8, T10, said T11, T4, T8 and T10 peaks having respective retention times at about 26.1 minutes (T11), about 111.5 minutes (T4), about 46.1 minutes (T8), and about 91.7 minutes (T10).

19. A botanical extract from the roots of plant Tripterygium Wilfordii Hook. F. having a HPLC profile, said HPLC profile comprising peaks of T1, T4, T8, T10, said T11, T4, T8 and T10 peaks having respective retention times at about 26.1 minutes (T11), about 111.5 minutes (T4), about 46.1 minutes (T8), and about 91.7 minutes (T10).

20. A botanical extract from the roots of plant Tripterygium Wilfordii Hook. F. having a HPLC profile, said HPLC profile comprising peaks T11, T4, T8 and T10, wherein said T11, T4, T8 and T10 peaks have respective retention times, relative to the retention time of a hydrocortisone peak, at about 0.23 (T11), 0.97 (T4), about 0.40 (T8), and about 0.80 (T10).

21. A botanical extract from the roots of plant Tripterygium Wilfordii Hook. F. having a HPLC profile, said HPLC profile comprising peaks U1, U2, U3, U4, U5, U6, U7, U8, and U9, said U1, U2, U3, U4, U5, U6, U7, U8, and U9 peaks having respective retention times, relative to the retention time of a hydrocortisone peak, at about 0.12 (U1), about 0.13(U2), 0.15(U3), about 0.34(U4), about 0.39(U5), about 0.45(U6), about 0.49(U7), about 1.26(U8), and about 1.28(U9).

22. The botanical extract in claim 21, further comprising peaks of T11, T4, T8 and T10, said T11, T4, T8 and T10 peaks have respective retention times, relative to the retention time of a hydrocortisone peak, at about 0.23 (T11), 0.97 (T4), about 0.40 (T8), and about 0.80 (T10).

23. A method of preparing a botanical extract from the roots of plant Tripterygium Wilfordii Hook. F., comprising the steps of
a) selecting said roots;
b) extracting said roots with an alcohol to produce an alcohol extract;
c) extracting said alcohol extract with chloroform to produce a chloroform extract;
d) applying said chloroform extract to a chromatographic column;
e) eluting said chromatographic column with a gradient chloroform solution and collecting fractions eluted from said column;
f) combining fractions containing triptriolide, tripdiolide and tripchloride; and
g) removing chloroform from said combined fractions to obtain said botanical extract.

24. The method of claim 23, wherein said alcohol is ethanol.

25. The method of claim 23, wherein said gradient chloroform solution comprises chloroform and ethanol.

26. The method of claim 23, further comprising the step of testing said fractions eluted from said chromatographic column to determine the presence of triptriolide, tripdiolide and tripchloride.

27. The method of claim 26, wherein said fractions are tested using thin layer chromatography.

28. A botanical extract from the roots of plant Tripterygium Wilfordii Hook. F. produced according to a process in claim 23.

29. A botanical extract from the roots of plant Tripterygium Wilfordii Hook. F. produced according to a process in claim 27.

30. A method of producing a HPLC fingerprint of a botanical extract from the roots of plant Tripterygium Wilfordii Hook. F., comprising the steps of:
a) preparing a HPLC sample containing said botanical extract in a sample solvent to produce a sample solution;
b) applying said sample solution onto a HPLC column;
c) applying a gradient solution to produce a HPLC profile; and
d) recording said HPLC profile to produce said botanical extract fingerprint.

31. The method of claim 30, wherein said gradient solution comprises methanol, acetonitrile, a phosphoric acid solution or a mixture thereof.

32. The method of claim 31, wherein said gradient solution comprises a solution A and a solution B, said solution B being applied to said HPLC column during predetermined time periods and at predetermined concentrations corresponding said respective predetermined time periods.

33. The method in claim 32, wherein said solution A comprises acetonitrile and said solution B comprises a 0.2% (v/v) of H₃PO₄ water solution.

34. The method in claim 33, wherein said solution B is applied to said HPLC column at a concentration of about 93% at the beginning followed by applying about 80% of said solution B after about 120 minutes from the beginning, applying about 70% of said solution B after about 160.00 from the beginning, and applying 0% of said solution B after about 161.00 minutes from the beginning.

35. The method in claim 30, wherein said HPLC column is a Water Symmetry C18 column having a demension of 4.6×250 mm and a particle size of 5μ.

36. The method in claim 30, wherein said sample solvent comprises methanol, 0.2% v/v H₃PO₄, acetonitrile and HPLC grade water.

37. The method in claim 36, wherein said methanol, said 0.2% V/V H₃PO₄, said acetonitrile and said HPLC grade water are mixed at a ratio of 65 (methanol):10 (0.2% v/v H₃PO₄):5 (acetonitrile):20 (HPLC grade water).

38. The method in claim 30, wherein said HPLC profile is recorded at a UV wavelength of 214 nm.

39. The method of claim 30, further comprising the step of employing hydrocortisone as an internal reference.

40. The method of claim 30, wherein said botanical extract is produced by the process of claim 23.

41. The method of claim 30, wherein said botanical extract is produced by the process of claim 24.

42. The method of claim 30, wherein said botanical extract is prepared by the process of claim 25.

43. The method of claim 30, wherein said botanical extract is obtained by the process of claim 26.

44. The method of claim 30, wherein said botanical extract is obtained by the process of claim 27.

45. A method of determining the amount of triptolide (T10) in a botanical extract from the roots of plant Tripterygium Wilfordii Hook. F., comprising the steps of:
a) preparing a plurality of T10 standards having predetermined concentrations in a sample solvent to produce a plurality of T10 standards solutions;
b) applying said plurality of T10 standard solutions to a HPLC column, respectively;
c) applying a gradient solution to said HPLC column to produce a plurality of area responses of T10 corresponding to said concentrations of said plurality of T10 standards;
d) plotting said area responses against said corresponding concentration of T10 to produce a standard plot;
e) preparing a HPLC sample containing said botanical extract in a sample solvent to produce a sample solution;
f) applying said sample solution onto said HPLC column;
g) applying said gradient solution to said HPLC column to produce a HPLC profile comprising a T10 peak; and
h) referring the area under said T10 peak to said standard plot to determine the amount of T10 in said botanical extract.

46. The method of claim 45, wherein said gradient solution comprises methanol, acetonitrile, a phosphoric acid solution or a mixture thereof.

47. The method in claim 46, wherein said gradient solution comprises a solution A and a solution B, said solution B being applied to said HPLC column during predetermined time periods and at predetermined concentrations corresponding to said predetermined time periods to produce a HPLC profile comprising a T10 peak.

48. The method in claim 47, wherein said solution A comprising a 0.2% (v/v) H₂PO₄ water solution and said solution B comprising acetonitrile.

49. The method in claim 48, wherein said sample solvent is methanol.

50. The method in claim 49, wherein said solution B is applied to said HPLC column at a concentration of about 19% at the beginning followed by applying about 100% of said solution B after about 28 minutes from the beginning, applying about 19% of said solution B after about 34 minutes from the beginning, and applying 0% of said solution B after about 45 minutes from the beginning.

51. The method of claim 45, wherein said botanical extract is obtained by the process of claim 23.

52. The method of claim 45, wherein said botanical extract is obtained by the process of claim 24.

53. The method of claim 45, wherein said botanical extract is prepared by the process of claim 25.

54. The method of claim 45, wherein said botanical extract is obtained by the process of claim 26.

55. The method of claim 45, wherein said botanical extract is obtained by the process of claim 27.

56. A method of determining the amount of triptriolide (T11) in a botanical extract from the roots of plant Tripterygium Wilfordii Hook. F., comprising the steps of:
a) preparing a T11 standard having a predetermined concentration to produce a standard stock solution;
b) diluting said standard stock solution with a sample solvent to produce a standard dilution;
c) applying said T11 standard dilution to a HPLC column;
d) applying a gradient solution to produce a T11 peak corresponding to said concentration of said T11 standard dilution;
e) preparing a HPLC sample having a predetermined concentration of said botanical extract to produce a sample stock solution;
f) diluting said sample stock solution with said sample solvent to produce a sample dilution;
g) applying said sample dilution onto said HPLC column;
h) applying said gradient solution said HPLC column to produce a HPLC profile comprising a T11 peak; and
i) calculating the concentration of T11 in said botanical extract according to the following formula
$$T11 ( \% w / w ) = \frac{A Spl \times Std . wt . \times P \times Spl dilution}{A Std \times Std dilution \times Spl . wt .}$$
Wherein A Spl is the area under said T11 peak in said sample; Std. wt. is the weight of said T11 standard; Spl dilution is ______; A Std is the area under said T11 standard peak; Std dilution is ______; and P is the purity of said T11 standard.

56. The method of claim 55, wherein said gradient solution comprises methanol, acetonitrile, a phosphoric acid solution or a mixture thereof.

57. The method in claim 56, wherein said gradient solution comprises a solution A and a solution B, said gradient solution being applied to said HPLC column during predetermined time periods and at predetermined concentrations.

58. The method in claim 57, wherein said solution A comprise acetonitrile and said solution B comprises a 0.2% (v/v) ortho-phosphoric acid water solution.

59. The method in claim 58, wherein said sample solvent is a 0.2% (v/v) ortho-phosphoric acid water solution.

60. The method in claim 59, wherein said solution B is applied to said HPLC column at a concentration of about 93% at the beginning followed by applying about 80% of said solution B after about 120 minutes from the beginning, applying about 70% of said solution B after about 160 minutes from the beginning, applying about 93% of said solution B after about 161 minutes, and applying 0% of said solution B after about 170 minutes from the beginning.

61. The method of claim 55, wherein said botanical extract is obtained by the process of claim 23.

62. The method of claim 55, wherein said botanical extract is obtained by the process of claim 24.

63. The method of claim 55, wherein said botanical extract is prepared by the process of claim 25.

64. The method of claim 55, wherein said botanical extract is obtained by the process of claim 26.

65. The method of claim 55, wherein said botanical extract is obtained by the process of claim 27.
